(19) United States
(12) Patent Application Publication
(10) Pub. No.: US 2016/0340632 A1
(43) Pub. Date: Nov. 24, 2016

(54) CULTURING STATION FOR MICROFLUIDIC DEVICE

(71) Applicant: BERKELEY LIGHTS, INC., Emeryville, CA (US)

(72) Inventors: Keith J. Breinlinger, Novato, CA (US); Russell A. Newstrom, Fremont, CA (US); J. Tanner Nevill, El Cerrito, CA (US); Jason M. McEwen, El Cerrito, CA (US); David A. Weisbach, Oakland, CA (US)

(21) Appl. No.: 15/135,252

(22) Filed: Apr. 21, 2016

Related U.S. Application Data
(60) Provisional application No. 62/178,960, filed on Apr. 22, 2015.

(51) Int. Cl. C12M 1/00; C12M 1/34; C12M 3/06; C12M 3/00; C12M 1/04; C12M 1/26; C12M 1/12; C12M 1/24; C12M 1/02; C12M 1/36

(52) U.S. Cl. CPC C12M 23/58; C12M 41/22; C12M 41/44; C12M 23/38; C12M 23/16; C12M 23/20; C12M 29/10; C12M 23/50; C12M 41/48; C12M 23/26; C12M 23/24; C12M 41/06; C12M 33/00; C12M 23/34; C12M 37/00; C12M 23/08; C12M 41/46; C12M 23/22; C12M 23/44

(57) ABSTRACT

A station for culturing biological cells in a microfluidic device is provided. The station includes one or more thermally conductive mounting interfaces, each mounting interface configured for having a microfluidic device detachably mounted thereon; a thermal regulation system configured for controlling a temperature of microfluidic devices detachably mounted on the one or more mounting interfaces; and a media perfusion system configured to controllably and selectively dispense a flowable culturing media into microfluidic devices detachably mounted on the one or mounting interfaces.

### RELATED APPLICATION DATA

The present application claims the benefit under 35 U.S.C. §119 to U.S. provisional patent application Ser. No. 62/178,960, filed Apr. 22, 2015. The foregoing application is hereby incorporated by reference into the present application in its entirety.

### FIELD OF THE INVENTION

The present disclosure relates generally to the processing and culturing of biological cells using microfluidic devices.

### BACKGROUND

As the field of microfluidics continues to progress, microfluidic devices have become convenient platforms for processing and manipulating micro-objects, such as biological cells. Even so, the full potential of microfluidic devices, particularly as applied to the biological sciences, has yet to be realized. For example, while microfluidic devices have been applied to the analysis of biological cells, the culturing of such cells continues to be performed in tissue culture plates, which is time consuming and requires relatively large amounts of costly cell culturing media, disposable plastic dishes, microtiter plates, and the like.

### Summary

In accordance with the exemplary embodiments disclosed herein, a station for culturing biological cells in a microfluidic device is provided. The station includes one or more thermally conductive mounting interfaces (e.g., one, two, three, four, five, six, or more, mounting interfaces), each mounting interface configured for having a microfluidic device detachably mounted thereon. The station further includes a thermal regulation system configured for controlling a temperature of microfluidic devices detachably mounted on each of the one or more mounting interfaces, and a media perfusion system configured to controllably and selectively dispense flowable culturing media into microfluidic devices detachably mounted on each of the one or more mounting interfaces.

In various embodiments, the media perfusion system includes a pump having an input fluidically connected to a source of culturing media and an output, which may be the same as or different than the input. Perfusion of media (or other fluids or gases) can be performed by a perfusion network that fluidically connects the pump output with one or more perfusion lines, each perfusion line associated with a respective one of the one or more mounting interfaces. The perfusion lines can be configured to be fluidically connected to a fluid ingress port of a microfluidic device mounted on the respective mounting interface. A control system is configured to selectively operate the pump and the perfusion network to thereby selectively cause culturing media from the culturing media source to flow through a respective perfusion line at a controlled flow rate for a controlled period of time. In various embodiments, the control system is (or may be) programmed or otherwise configured to provide an intermittent flow of culturing media through a respective perfusion line according to an on-off duty cycle and a flow rate, which may optionally be based at least in part on input received through a user interface. In some embodiments, the control system is (or may be) programmed or otherwise configured to provide a flow of culturing media through no more than a single perfusion line at any one time. In other embodiments, the control system is (or may be) programmed or otherwise configured to provide a flow of culturing media through two or more perfusion lines at the same time.

In various embodiments, the culturing station further includes respective microfluidic device covers associated with each mounting interface, the device covers being configured to partially or fully enclose a microfluidic device mounted on the respective mounting interface. A perfusion line associated with the respective mounting interface can have a distal end coupled to the device cover, configured in conjunction with a configuration of the device cover so that the distal end of the perfusion line may be fluidically connected to a fluid ingress port on the microfluidic device when the device cover is enclosing (e.g., positioned over) the microfluidic device. For example, the device covers can include one or more features configured to form a pressure fit, a frictional fit, or another type of fluid tight connection between the distal end of the perfusion line and the fluid ingress port of the microfluidic device in order to fluidically connect the perfusion line to the microfluidic device.

One or more waste lines may also be associated with a respective one of the one or more mounting interfaces. For example, the respective waste lines can be coupled to each of the one or more device covers, each waste line having a proximal end coupled to the respective device cover and configured in conjunction with a configuration of the cover so that the proximal end of the waste line may be fluidically connected to a fluid egress port on the microfluidic device when the device cover is enclosing (e.g., positioned over) the microfluidic device. The device covers can include one or more features configured to form a pressure fit, a frictional fit, or another type of fluid tight connection between the proximal end of the waste line and the fluid egress port of the microfluidic device in order to fluidically connect the waste line to the microfluidic device.

In various embodiments, each mounting interface can comprise a generally planar metallic substrate having a top surface configured to thermally couple with a generally planar metallic bottom surface of a microfluidic device mounted thereon. The substrate can further comprise a bottom surface configured to thermally couple with a heating element, such as a resistive heater, a Peltier thermoelectric device, or the like. The substrate can comprise a copper alloy, such as brass or bronze.

The thermal regulation system can include one or more temperature sensors. Such sensors can be coupled to and/or embedded within each mounting interface substrate. Alternatively, or in addition, the thermal regulation system can be configured to receive temperature data from one or more temperature sensors coupled to and/or embedded within each microfluidic device mounted on a mounting interface. In one embodiment, the thermal regulation system can include one or more resistive heaters thermally coupled to the one or more mounting interfaces, optionally with each of the one or more resistive heaters being thermally coupled to a respective one of the one or more mounting interfaces or a metallic substrate thereof. In an alternate embodiment, the thermal regulation system can include one or more Peltier thermoelectric heating/cooling devices, optionally with each of the one or more Peltier devices being thermally coupled to a respective one of the one or more mounting interfaces or a metallic substrate thereof.

The thermal regulation system can comprising one or more printed circuit boards (PCBs) configured to monitor and regulate the temperature of the one or more mounting interfaces. Thus, the one or more PCBs can obtain temperature data from the one or more temperature sensors (whether coupled to and/or mounted on a mounting interface and/or a microfluidic device mounted thereon) and use such data to regulate the temperature of the one or more mounting interfaces and/or microfluidic devices mounted thereon. The one or more PCBs can comprise a resistive heater (e.g., a metal lead on the surface of the PCB that heats up when current is passed through) of can be coupled to a heating element, such as a resistive heater or a Peltier device. Each of the one or more printed circuit boards (PCBs) can be associated with a respective one of the one or more mounting interfaces. Thus, each of the one or more mounting interfaces can be independently monitored and regulated with regard to temperature.

In various embodiments, a respective adjustable clamp is provided at each mounting interface and configured to secure a microfluidic device to the respective mounting interface. For example, in embodiments in which device covers are provided at the mounting interfaces, the clamps may be configured to apply a force against the respective device cover associated with the mounting interface such that the device cover secures a microfluidic device at least partially enclosed by (e.g., positioned under) the device cover to the respective mounting surface. In other embodiments, one or more compression springs are provided at each mounting interfaces and configured to apply a force against a respective device cover associated with the mounting interface, such that the device cover secures a microfluidic device at least partially enclosed by the device cover to the respective mounting surface.

In various embodiments, the culturing station further comprises a support for the one or more mounting interfaces, the support being configured to rotate about a defined axis and thereby allow the one or more mounting interfaces to be tilted relative to a plane that is normal to the gravitational force acting upon the culturing station. In such embodiments, the culturing station can further include a level, which can indicate when the one or more mounting interfaces is/are tilted at a pre-determined degree relative to the normal plane, thus allowing microfluidic devices mounted on the mounting interfaces to be held at a desired angle. For example, the pre-determined degree of tilt can be within the range of about 0.5° to about 135° (e.g., about 1°, 2°, 3°, 4°, 5°, 10°, 15°, 20°, 25°, 30°, 35°, 40°, 45°, 50°, 55°, 60°, 65°, 70°, 75°, 80°, 85°, 90°, 95°, 100°, 105°, 110°, 115°, 120°, 125°, 130°, or 135°.

In various embodiments, the culturing station is further configured to record in a memory respective perfusion and/or temperature histories of microfluidic devices mounted to the one or more mounting interfaces. By way of non-limiting example, the memory can be incorporated into or otherwise coupled with the respective microfluidic device. The culturing station may further be equipped with an imaging and/or detecting apparatus coupled to or otherwise operatively associated with the culturing station and configured for viewing and/or imaging and/or detecting biological activity in a microfluidic device mounted to a mounting interface.

In accordance with another aspect of the disclosed embodiments, an exemplary method for culturing biological cells in a microfluidic device includes (i) mounting a microfluidic device on a mounting interface of a culturing station, the microfluidic device defining a microfluidic circuit including a flow region and a plurality of growth chambers, the microfluidic device comprising a fluid ingress port in fluid communication with a first end region of the microfluidic circuit, and a fluid egress port in fluid communication with a second end region of the microfluidic circuit; (ii) fluidically connecting a perfusion line associated with the mounting interface to the fluid ingress port to thereby fluidically connect the perfusion line with the first end region of the microfluidic circuit; (iii) fluidically connecting a waste line associated with the mounting interface to the fluid egress port to thereby fluidically connect the waste line with the second end region of the microfluidic circuit; and (iv) flowing a culturing media through the perfusion line, fluid ingress port, flow region of the microfluidic circuit, and fluid egress port, respectively, at a flow rate adequate to perfuse one or more biological cells sequestered in the plurality of growth chambers.

In various embodiments, an intermittent flow of culturing media is provided through the flow region of the microfluidic circuit. By way of example, the culturing media can be flowed through the flow region of the microfluidic circuit according to a predetermined and/or operator selected on-off duty cycle, which may (without limitation), last for about 5 minutes to about 30 minutes (e.g., about 5 minutes to about 10 minutes, about 6 minutes to about 15 minutes, about 7 minutes to about 20 minutes, about 8 minutes to about 25 minutes, about 15 minutes to about 20, 25, or 30 minutes, about 17.5 minutes to about 20, 25, or 30 minutes. In some embodiments, culturing media is flowed periodically, each time (by way of example and not limitation) for about 10 seconds to about 120 seconds (e.g., about 20 seconds to about 100 seconds, or about 30 seconds to about 80 seconds). In some embodiments, flow of culturing media in the flow region of the microfluidic circuit is stopped periodically (by way of example and not limitation) for about 5 seconds to about 60 minutes (e.g., about 30 seconds to about 1, 2, 3, 4, 5, or 30 minutes, about 1 minute to about 2, 3, 4, 5, 6, or 35 minutes, about 2 minutes to about 4, 5, 6, 7, 8, or 40 minutes, about 3 minutes to about 6, 7, 8, 9, 10, or 45 minutes, about 4 minutes to about 8, 9, 10, 11, 12, or 50 minutes, about 5 minutes to about 10, 15, 20, 25, 30, or 60 minutes, about 10 minutes to about 20, 30, 40, 50, or 60 minutes, etc.). The culturing media can be flowed through the flow region of the microfluidic circuit according to a predetermined and/or operator selected flow rate. By way of non-limiting example, in one embodiment, the flow rate is about 0.01 microliters/sec to about 5.0 microliters/sec. In various embodiments, the flow region of the microfluidic circuit comprises two or more flow channels, wherein the culturing media is flowed through each of the two or more flow channels at an average rate of (again, by way of example and not limitation) about 0.005 microliters/sec to about 2.5 microliters/sec. In alternative embodiments, a continuous flow of culturing media is provided through the microfluidic circuit.

In various embodiments, the method further includes controlling a temperature of the microfluidic device using at least one heating element (e.g., a resistive heater, a Peltier thermoelectric device, or the like) that is thermally coupled to the mounting interface. For example, the heating element can be activated based on a signal output by a temperature sensor embedded in or otherwise coupled to the mounting interface.

In various embodiments, the method further includes recording perfusion and/or temperature histories of the microfluidic device while it is mounted to the mounting interface. By way of non-limiting example, the perfusion and/or temperature histories can be recorded in a memory that is incorporated into or otherwise coupled to the microfluidic device.

Other and further aspects and features of embodiments of the disclosed inventions will become apparent from the ensuing detailed description in view of the accompanying figures.

### BRIEF DESCRIPTION OF THE DRAWINGS

FIG. 1A is a perspective view of an exemplary embodiment of a system including a microfluidic device for culturing biological cells.

FIG. 1B is a side, cross-sectional view of the microfluidic device of FIG. 1A.

FIG. 1C is a top, cross-sectional view of the microfluidic device of FIG. 1A.

FIG. 1D is side cross-sectional view of an embodiment of a microfluidic device having a dielectrophoresis (DEP) configuration.

FIG. 1E is a top, cross-sectional view of one embodiment of the microfluidic device of FIG. 1D.

FIG. 2 illustrates an example of a growth chamber that may be used in the microfluidic device of FIG. 1A, in which a length of a connection region from a flow channel to an isolation region is greater than a penetration depth of medium flowing in the flow channel.

FIG. 3 is another example of a growth chamber that may be used in the microfluidic device of FIG. 1A, including a connection region from a flow channel to an isolation region that is longer than a penetration depth of medium flowing in the flow channel.

FIGS. 4A-C show another embodiment of a microfluidic device, including a further example of a growth chamber used therein.

FIG. 5 is a perspective view of a pair of culturing stations shown in a side-by-side arrangement, according to one embodiment, each of the culturing stations having a single thermally regulated microfluidic device mounting interface.

FIG. 6 is a perspective view of a mounting interface of one of the culturing stations of FIG. 5, depicting a microfluidic device cover that covers a mounting surface thereof.

FIG. 7 is a perspective view of the mounting interface shown in FIG. 6, with the microfluidic device cover removed to reveal the mounting interface surface.

FIG. 8 is a perspective view of the mounting interface shown in FIG. 6, depicting a respective microfluidic device and microfluidic device cover mounted thereon.

FIG. 9 is a side view of the mounting interface shown in FIG. 6, depicting components of a thermal regulation system.

FIG. 10 is a perspective view of another embodiment of a culturing station for culturing biological cells in microfluidic devices, including a support (or tray) having six thermally regulated mounting interfaces and a media perfusion system having two pumps, each configured to service three microfluidic devices.

FIG. 11 is a perspective view of a portion of the support and associated mounting interfaces shown in FIG. 10, depicting respective microfluidic device covers and clamps associated with their respective mounting interfaces.

FIG. 12 is a perspective view of one of the mounting interfaces of the support shown in FIG. 10, with the microfluidic device cover removed and the clamp raised to reveal the mounting interface surface.

FIG. 13 is a perspective view of an alternate support (or tray) having five thermally regulated mounting interfaces for use with the culturing station of FIG. 10.

FIG. 14 is a perspective view of a mounting interface of the tray shown in FIG. 13, depicting a microfluidic device cover that encloses a microfluidic device mounted thereon.

FIG. 15 is a perspective view of the mounting interface of FIG. 14, wherein the microfluidic device cover is removed to show the microfluidic device mounted thereon.

### DETAILED DESCRIPTION

This specification describes exemplary embodiments and applications of the invention. The invention, however, is not limited to these exemplary embodiments and applications or to the manner in which the exemplary embodiments and applications operate or are described herein. Moreover, the Figures may show simplified or partial views, and the dimensions of elements in the Figures may be exaggerated or otherwise not in proportion for clarity. In addition, as the terms “on,” “attached to,” or “coupled to” are used herein, one element (e.g., a material, a layer, a substrate, etc.) can be “on,” “attached to,” or “coupled to” another element regardless of whether the one element is directly on, attached, or coupled to the other element or there are one or more intervening elements between the one element and the other element. Also, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements.

Section divisions in the specification are for ease of review only and do not limit any combination of elements discussed.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent. The term “ones” means more than one.

As used herein, the term “micro-object” can encompass one or more of the following: inanimate micro-objects such as microparticles, microbeads (e.g., polystyrene beads, Luminex™ beads, or the like), magnetic beads, paramagnetic beads, microrods, microwires, quantum dots, and the like; biological micro-objects such as cells (e.g., embryos, oocytes, sperms, cells dissociated from a tissue, blood cells, immunological cells, such as macrophages, NK cells, T cells, B cells, dendritic cells (DCs), and the like, hybridomas, cultured cells, cells dissociated from a tissue, cells from a cell line, such as CHO cells, cancer cells, circulating tumor cells (CTCs), infected cells, transfected and/or transformed cells, reporter cells, and the like), liposomes (e.g., synthetic or derived from membrane preparations), lipid nanorafts, and the like; or a combination of inanimate micro-objects and biological micro-objects (e.g., microbeads attached to cells, liposome-coated micro-beads, liposome-coated magnetic beads, or the like). Lipid nanorafts have been described, e.g., in Ritchie et al. (2009) “Reconstitution of Membrane Proteins in Phospholipid Bilayer Nanodiscs,” Methods Enzymol., 464:211-231.

As used herein, the term “cell” refers to a biological cell, which can be a plant cell, an animal cell (e.g., a mammalian cell), a bacterial cell, a fungal cell, or the like. A mammalian cell can be, for example, from a human, a mouse, a rat, a horse, a goat, a sheep, a cow, a primate, or the like.

As used herein, the term “maintaining (a) cell(s)” refers to providing an environment comprising both fluidic and gaseous components and, optionally a surface, that provides the conditions necessary to keep the cells viable and/or expanding.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein in reference to a fluidic medium, “diffuse” and “diffusion” refer to thermodynamic movement of a component of the fluidic medium down a concentration gradient.

The phrase “flow of a medium” means bulk movement of a fluidic medium primarily due to any mechanism other than diffusion. For example, flow of a medium can involve movement of the fluidic medium from one point to another point due to a pressure differential between the points. Such flow can include a continuous, pulsed, periodic, random, intermittent, or reciprocating flow of the liquid, or any combination thereof. When one fluidic medium flows into another fluidic medium, turbulence and mixing of the media can result.

The phrase “substantially no flow” refers to a rate of flow of a fluidic medium that, averaged over time, is less than the rate of diffusion of components of a material (e.g., an analyte of interest) into or within the fluidic medium. The rate of diffusion of components of such a material can depend on, for example, temperature, the size of the components, and the strength of interactions between the components and the fluidic medium.

As used herein in reference to different regions within a microfluidic device, the phrase “fluidically connected” means that, when the different regions are substantially filled with fluid, such as fluidic media, the fluid in each of the regions is connected so as to form a single body of fluid. This does not mean that the fluids (or fluidic media) in the different regions are necessarily identical in composition. Rather, the fluids in different fluidically connected regions of a microfluidic device can have different compositions (e.g., different concentrations of solutes, such as proteins, carbohydrates, ions, or other molecules) which are in flux as solutes move down their respective concentration gradients and/or fluids flow through the device.

In some embodiments, a microfluidic device can comprise “swept” regions and “unswept” regions. An unswept region can be fluidically connected to a swept region, provided the fluidic connections are structured to enable diffusion but substantially no flow of media between the swept region and the unswept region. The microfluidic device can thus be structured to substantially isolate an unswept region from a flow of medium in a swept region, while enabling substantially only diffusive fluidic communication between the swept region and the unswept region.

A “microfluidic channel” or “flow channel” as used herein refers to flow region of a microfluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 300 times the length, at least 400 times the length, at least 500 times the length, or longer. In some embodiments, the length of a flow channel is in the range of from about 20,000 microns to about 100,000 microns, including any range therebetween. In some embodiments, the horizontal dimension is in the range of from about 100 microns to about 300 microns (e.g., about 200 microns) and the vertical dimension is in the range of from about 25 microns to about 150 microns, e.g., from about 30 to about 100 microns, or about 40 to about 60 microns. It is noted that a flow channel may have a variety of different spatial configurations in a microfluidic device, and thus is not restricted to a perfectly linear element. For example, a flow channel may be, or include one or more sections having, the following configurations: curve, bend, spiral, incline, decline, fork (e.g., multiple different flow paths), and any combination thereof. In addition, a flow channel may have different cross-sectional areas along its path, widening and constricting to provide a desired fluid flow therein.

In certain embodiments, a flow channel of a micro-fluidic device is an example of a swept region (defined above) while an isolation region (described in further detail below) of a microfluidic device is an example of an unswept region.

The capability of biological micro-objects (e.g., biological cells) to produce specific biological materials (e.g., proteins, such as antibodies) can be assayed in such a microfluidic device. For example, sample material comprising biological micro-objects (e.g., cells) to be assayed for production of an analyte of interest can be loaded into a swept region of the microfluidic device. Ones of the biological micro-objects (e.g., mammalian cells, such as human cells) can be selected for particular characteristics and disposed in unswept regions. The remaining sample material can then be flowed out of the swept region and an assay material flowed into the swept region. Because the selected biological micro-objects are in unswept regions, the selected biological micro-objects are not substantially affected by the flowing out of the remaining sample material or the flowing in of the assay material. The selected biological micro-objects can be allowed to produce the analyte of interest, which can diffuse from the unswept regions into the swept region, where the analyte of interest can react with the assay material to produce localized detectable reactions, each of which can be correlated to a particular unswept region. Any unswept region associated with a detected reaction can be analyzed to determine which, if any, of the biological micro-objects in the unswept region are sufficient producers of the analyte of interest.

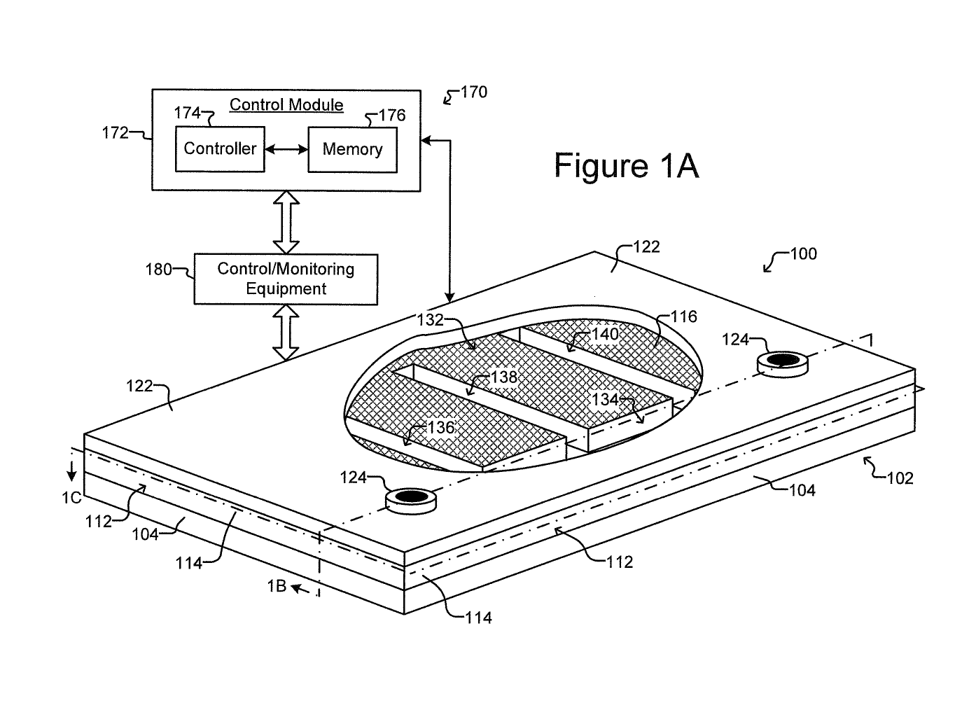

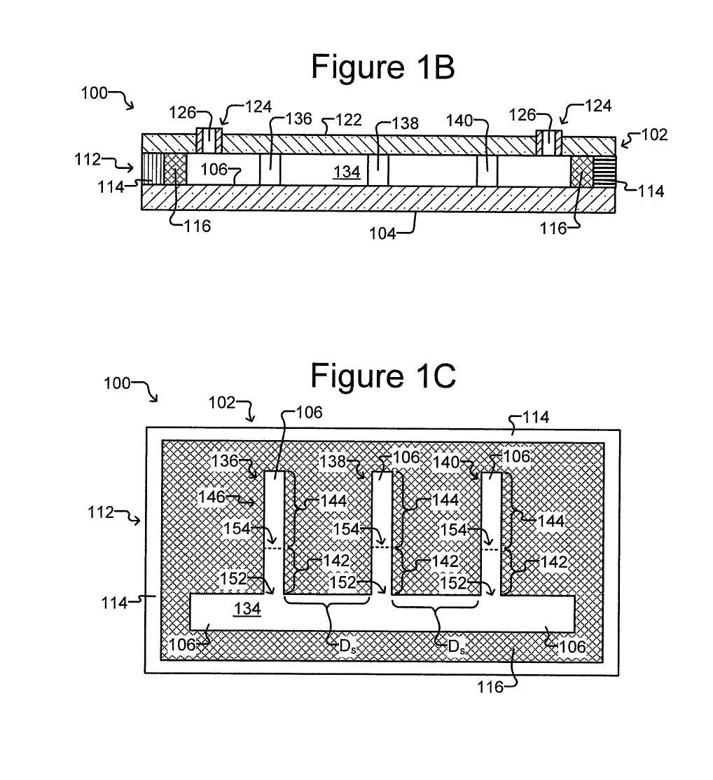

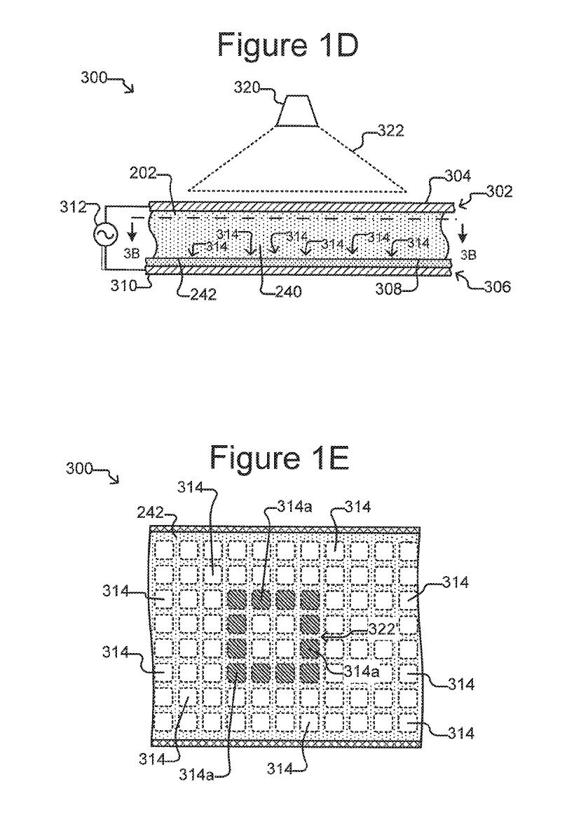

System including a microfluidic device. FIGS. 1A-1C illustrate an example of a system having a microfluidic device 100 which may be used in the methods described herein. As shown, the microfluidic device 100 encloses a microfluidic circuit 132 comprising a plurality of interconnected fluidic circuit elements. In the example illustrated in FIGS. 1A-1C, the microfluidic circuit 132 includes a flow channel 134 to which growth chambers 136, 138, 140 are fluidically connected. Although one flow channel 134 and three growth chambers 136, 138, 140 are shown in the illustrated embodiment, it should be understood that there may be more than one flow channel 134, and more or fewer than three growth chambers 136, 138, 140, respectively, in alternate embodiments. The microfluidic circuit 132 can also include additional or different fluidic circuit elements such as fluidic chambers, reservoirs, and the like.

The microfluidic device 100 comprises an enclosure 102 enclosing the microfluidic circuit 132, which can contain one or more fluidic media. Although the device 100 can be physically structured in different ways, in the embodiment shown in FIGS. 1A-1C, the enclosure 102 includes a support structure 104 (e.g., a base), a microfluidic circuit structure 112, and a cover 122. The support structure 104, microfluidic circuit structure 112, and the cover 122 can be attached to each other. For example, the microfluidic circuit structure 112 can be disposed on the support structure 104, and the cover 122 can be disposed over the microfluidic circuit structure 112. With the support structure 104 and the cover 122, the microfluidic circuit structure 112 can define the microfluidic circuit 132. An inner surface of the microfluidic circuit 132 is identified in the figures as 106.

The support structure 104 can be at the bottom and the cover 122 at the top of the device 100 as illustrated in FIGS. 1A and 1B. Alternatively, the support structure 104 and cover 122 can be in other orientations. For example, the support structure 104 can be at the top and the cover 122 at the bottom of the device 100. Regardless of the configuration, one or more fluid access (i.e., ingress and egress) ports 124 are provided, each fluid access port 124 comprising a passage 126 in communication with the microfluidic circuit 132, which allow for a fluid material to be flowed into, or out of, the enclosure 102. The fluid passages 126 may include a valve, a gate, a pass-through hole, or the like. Although two fluid access ports 124 are shown in the illustrated embodiment, it should be understood that alternate embodiments of the device 100 can have only one or more than two fluid access ports 124 providing ingress and egress of fluid material into and out of the microfluidic circuit 132.

The microfluidic circuit structure 112 can define or otherwise accommodate circuit elements of the microfluidic circuit 132, or other types of circuits located within the enclosure 102. In the embodiment illustrated in FIGS. 1A-1C, the microfluidic circuit structure 112 comprises a frame 114 and a microfluidic circuit material 116.

The support structure 104 can comprise a substrate or a plurality of interconnected substrates. For example, the support structure 104 can comprise one or more interconnected semiconductor substrates, printed circuit boards (PCB), or the like, and combinations thereof (e.g. a semiconductor substrate mounted on a PCB). The frame 114 can partially or completely enclose the microfluidic circuit material 116. The frame 114 can be, for example, a relatively rigid structure substantially surrounding the microfluidic circuit material 116. For example the frame 114 can comprise a metal material.

The microfluidic circuit material 116 can be patterned with cavities or the like to define microfluidic circuit elements and interconnections of the microfluidic circuit 132. The microfluidic circuit material 116 can comprise a flexible material (e.g. a rubber, plastic, elastomer, silicone or organosilicone polymer, such as polydimethylsiloxane (“PDMS”), or the like), which can be gas permeable. Other examples of materials that can compose microfluidic circuit material 116 include molded glass, an etchable material such as silicone (e.g. photo-patternable silicone), photo-resist (e.g., an epoxy-based photo-resist, such as SU8), or the like. In some embodiments, such materials—and thus the microfluidic circuit material 116—can be rigid and/or substantially impermeable to gas. Regardless of the material(s) used, the microfluidic circuit material 116 is disposed on the support structure 104, within the frame 114.

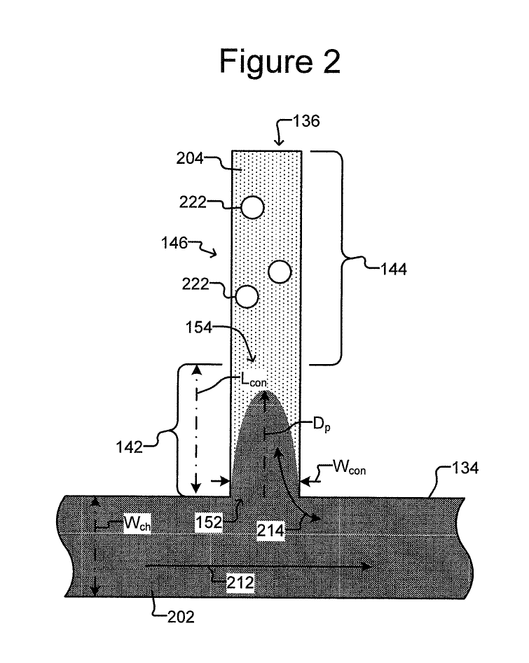

The cover 122 can be an integral part of the frame 114 and/or the microfluidic circuit material 116. Alternatively, the cover 122 can be a structurally distinct element (as illustrated in FIGS. 1A and 1B). The cover 122 can comprise the same or different materials than the frame 114 and/or the microfluidic circuit material 116. Similarly, the support structure 104 can be a separate structure from the frame 114 or microfluidic circuit material 116, as illustrated, or an integral part of the frame 114 or microfluidic circuit material 116. Likewise the frame 114 and microfluidic circuit material 116 can be separate structures as shown in FIGS. 1A-1C or integral portions of the same structure. In some embodiments, the cover or lid 122 is made from a rigid material. The rigid materials may be glass or the like. In some embodiments, the rigid material may be conductive (e.g. ITO-coated glass) and/or modified to support cell adhesion, viability and/or growth. The modification may include a coating of a synthetic or natural polymer. In some embodiments, a portion of the cover or lid 122 that is positioned over a respective growth chamber 136, 138, 140 of FIGS. 1A-1C, or the equivalent in the below-described embodiments illustrated in FIGS. 2, 3, and 4, is made of a deformable material, including but not limited to PDMS. Thus the cover or lid 122 may be a composite structure having both rigid and deformable portions. In some embodiments, the cover 122 and/or the support structure 104 is transparent to light.

The cover 122 may also include at least one material that is gas permeable, including but not limited to PDMS.

Other system components. FIG. 1A also illustrates simplified block diagram depictions of a control/monitoring system 170 that can be utilized in conjunction with the microfluidic device 100, which together provide a system for biological cell culturing. As shown (schematically), the control/monitoring system 170 includes a control module 172 and control/monitoring equipment 180. The control module 172 can be configured to control and monitor the device 100 directly and/or through the control/monitoring equipment 180.

The control module 172 includes a controller 174 and a memory 176. The controller 174 can be, for example, a digital processor, computer, or the like, and the memory 176 can be, for example, a non-transitory digital memory for storing data and machine executable instructions (e.g., software, firmware, microcode, or the like) as non-transitory data or signals. The controller 174 can be configured to operate in accordance with such machine executable instructions stored in the memory 176. Alternatively or in addition, the controller 174 can comprise hardwired digital circuitry and/or analog circuitry. The control module 172 can thus be configured to perform (either automatically or based on user-directed input) any process useful in the methods described herein, step of such a process, function, act, or the like discussed herein.

The control/monitoring equipment 180 can comprise any of a number of different types of devices for controlling or monitoring the microfluidic device 100 and processes performed with the microfluidic device 100. For example, the control/monitoring equipment 180 can include power sources (not shown) for providing power to the microfluidic device 100; fluidic media sources (not shown) for providing fluidic media to or removing media from the microfluidic device 100; motive modules such as, by way of non-limiting example, a selector control module (described below) for controlling selection and movement of micro-objects (not shown) in the microfluidic circuit 132; image capture mechanisms such as, by way of non-limiting example, a detector (described below) for capturing images (e.g., of micro-objects) inside the microfluidic circuit 132; stimulation mechanisms such as, by way of non-limiting example, the below-described light source 320 of the embodiment illustrated in FIG. 1D, for directing energy into the microfluidic circuit 132 to stimulate reactions; and the like.

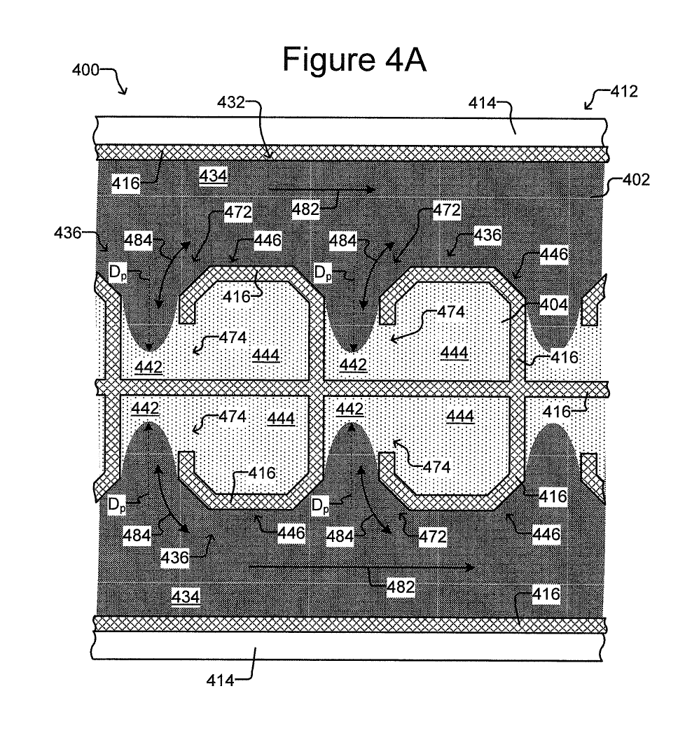

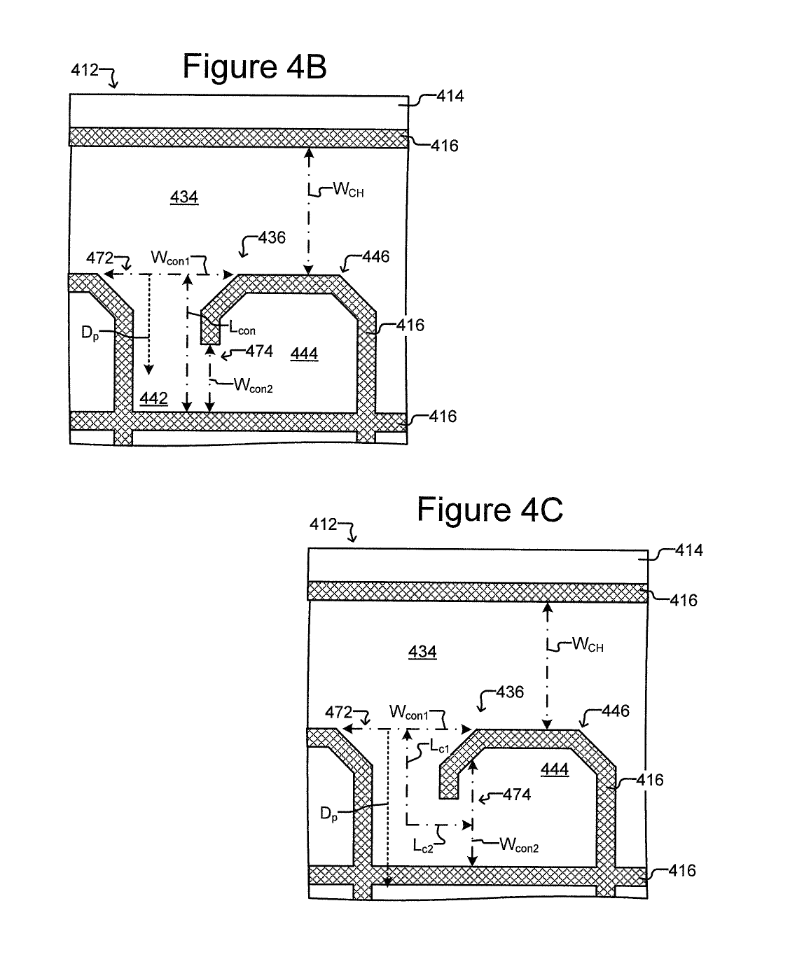

More particularly, an image capture detector can include one or more image capture devices and/or mechanisms for detecting events in the flow regions, including but not limited to flow channel 134 of the embodiments shown in FIGS. 1A-1C, 2, and 3, flow channel 434 of the embodiment shown in FIGS. 4A-4C, and flow region 240 of the embodiment shown in FIG. 1D-1E, and/or the growth chambers of the respective illustrated microfluidic devices 100, 300, and 400, including micro-objects contained in a fluidic medium occupying the respective flow regions and/or growth chambers. For example, the detector can comprise a photodetector capable of detecting one or more radiation characteristics (e.g., due to fluorescence or luminescence) of a micro-object (not shown) in the fluidic medium. Such a detector can be configured to detect, for example, that one or more micro-objects (not shown) in the medium are radiating electromagnetic radiation and/or the approximate wavelength, brightness, intensity, or the like of the radiation. The detector may capture images under visible, infrared, or ultraviolet wavelengths of light. Examples of suitable photodetectors include without limitation photomultiplier tube detectors and avalanche photodetectors.

Examples of suitable imaging devices that the detector can comprise include digital cameras or photosensors such as charge coupled devices and complementary metal-oxide-semiconductor (CMOS) imagers. Images can be captured with such devices and analyzed (e.g., by the control module 172 and/or a human operator).

A flow controller can be configured to control a flow of the fluidic medium in the flow regions/flow channels/swept regions of the respective illustrated microfluidic devices 100, 300, and 400. For example, the flow controller can control the direction and/or velocity of the flow. Non-limiting examples of such flow control elements of the flow controller include pumps and fluid actuators. In some embodiments, the flow controller can include additional elements such as one or more sensors for sensing, for example, the velocity of the flow and/or the pH of the medium in the flow region/flow channel/swept region.

The control module 172 can be configured to receive signals from and control the selector control module, the detector, and/or the flow controller.

Referring in particular to the embodiment shown in FIG. 1D, a light source 320 may direct light useful for illumination and/or fluorescent excitation into the microfluidic circuit 132. Alternatively, or in addition, the light source may direct energy into the microfluidic circuit 132 to stimulate reactions which include providing activation energy needed for DEP configured microfluidic devices to select and move micro-objects. The light source may be any suitable light source capable of projecting light energy into the microfluidic circuit 132, such as a high pressure Mercury lamp, Xenon arc lamp, diode, laser or the like. The diode may be an LED. In one non-limiting example the LED may be a broad spectrum “white” light LED (e.g. a UHP-T-LED-White by Prizmatix). The light source may include a projector or other device for generating structured light, such as a digital micromirror device (DMD), a MSA (microarray system) or a laser.

Motive modules for selecting and moving micro-objects including biological cells. As described above, the control/monitoring equipment 180 can comprise motive modules for selecting and moving micro-objects (not shown) in the microfluidic circuit 132. A variety of motive mechanisms can be utilized. For example, dielectrophoresis (DEP) mechanisms can be utilized to select and move micro-objects (not shown) in the microfluidic circuit. The support structure 104 and/or cover 122 of the microfluidic device 100 of FIGS. 1A-1C can comprise DEP configurations for selectively inducing DEP forces on micro-objects (not shown) in a fluidic medium (not shown) in the microfluidic circuit 132 and thereby select, capture, and/or move individual micro-objects. The control/monitoring equipment 180 can include one or more control modules for such DEP configurations. Micro-objects, including cells, may alternatively be moved within the microfluidic circuit or exported from the microfluidic circuit using gravity, magnetic force, fluid flow and/or the like.

One example of a microfluidic device having a DEP configuration that comprises support structure 104 and cover 122 is the microfluidic device 300 illustrated in FIG. 1D and 1E. While for purposes of simplicity FIGS. 1D and 1E show a side cross-sectional view and a top cross-sectional view of a portion of a flow region 240 of the microfluidic device 300, it should be understood that the microfluidic device 300 may also include one or more growth chambers, as well as one or more additional flow regions/flow channels, such as those described herein with respect to microfluidic devices 100 and 400, and that a DEP configuration may be incorporated in any of such regions of the microfluidic device 300. It should be further appreciated that any of the above or below described microfluidic system components may be incorporated in and/or used in combination with microfluidic device 300. For example, a control module 172 including control/monitoring equipment 180 described above in conjunction with microfluidic device 100 of FIGS. 1A-1C may also be used with the microfluidic device 300, including one or more of an image-capture detector, flow controller, and selector control module.

As seen in FIG. 1D, the microfluidic device 300 includes a first electrode 304, a second electrode 310 spaced apart from the first electrode 304, and an electrode activation substrate 308 overlying electrode 310. The respective first electrode 304 and electrode activation substrate 308 define opposing surfaces of the flow region 240, wherein a medium 202 contained in the flow region 240 provides a resistive flow path between electrode 304 and the electrode activation substrate 308. A power source 312 configured to be connected to the first electrode 304 and the second electrode 310 and create a biasing voltage between the electrodes, as required for the generation of DEP forces in the flow region 240, is also shown. The power source 312 can be, for example, an alternating current (AC) power source.

In certain embodiments, the microfluidic device 300 illustrated in FIGS. 1D and 1E can have an optically-actuated DEP configuration, such as an Opto-Electronic Tweezer (OET) configuration. In such embodiments, changing patterns of light 322 from the light source 320, which may be controlled by the selector control module, can be used to selectively activate changing patterns of “DEP electrodes” on targeted locations 314 on the inner surface 242 of the flow region 240. Hereinafter the targeted regions 314 on the inner surface 242 of the flow region 240 are referred to as “DEP electrode regions.”

In the example illustrated in FIG. 1E, a light pattern 322′ directed onto the inner surface 242 illuminates the cross-hatched DEP electrode regions 314a in the square pattern shown. The other DEP electrode regions 314 are not illuminated and are hereinafter referred to as “dark” DEP electrode regions 314. The electrical impedance through the DEP electrode activation substrate 308 (i.e., from each dark electrode region 314 on the inner surface 242 to the second electrode 310) is greater than the electrical impedance through the medium 202 (i.e., from the first electrode 304, across the medium 202 in the flow region 240, to the dark DEP electrode regions 314 on the inner surface 242). Illuminating the DEP electrode regions 314a, however, reduces the impedance through the electrode activation substrate 308 (i.e., from the illuminated DEP electrode regions 314a on the inner surface 242 to the second electrode 310) to less than the impedance through the medium 202 (i.e., from the first electrode 304, across the medium 202 in the flow region 240, to the illuminated DEP electrode regions 314a on the inner surface 242).

With the power source 312 activated, the foregoing creates an electric field gradient in the medium 202 between the respective illuminated DEP electrode regions 314a and adjacent dark DEP electrode regions 314, which in turn creates localized DEP forces that attract or repel nearby micro-objects (not shown) in the fluid medium 202. In this manner, DEP electrodes that attract or repel micro-objects in the medium 202 can be selectively activated and deactivated in order to manipulate, i.e., move, the micro-objects within the flow region 240 by changing the light patterns 322 projected from the light source 320 into the microfluidic device 300. The light source 320 can be, for example, a laser or other type of structured light source, such as a projector. Whether the DEP forces attract or repel nearby micro-objects can depend on parameters such as, without limitation, the frequency of the power source 312 and the dielectric properties of the medium 202 and/or micro-objects (not shown).

The square pattern 322′ of illuminated DEP electrode regions 314a illustrated in FIG. 1E is an example only. Any number of patterns or configurations of DEP electrode regions 314 can be selectively illuminated by a corresponding pattern of light 322 projected from the source 320 into the device 300, and the pattern of illuminated DEP electrode regions 322′ can be repeatedly changed by changing the light pattern 322 in order to manipulate micro-objects in the fluid medium 202.

In some embodiments, the electrode activation substrate 308 can be a photoconductive material, and the rest of the inner surface 242 can be featureless. For example, the photoconductive material can be made from amorphous silicon, and can form a layer having a thickness of about 500 nm to about 2 μm in thickness (e.g. substantially 1 micron in thickness). In such embodiments, the DEP electrode regions 314 can be created anywhere and in any pattern on the inner surface 242 of the flow region 240 in accordance with the light pattern 322 (e.g., light pattern 322′ shown in FIG. 1E). The number and pattern of the illuminated DEP electrode regions 314a are thus not fixed, but correspond to the respective projected light patterns 322. Examples are illustrated in U.S. Pat. No. 7,612,355, in which un-doped amorphous silicon material is used as an example of photoconductive material that can compose the electrode activation substrate 308.

In other embodiments, the electrode activation substrate 308 can comprise a substrate comprising a plurality of doped layers, electrically insulating layers, and electrically conductive layers that form semiconductor integrated circuits such as is known in semiconductor fields. For example, the electrode activation substrate 308 can comprise an array of photo-transistors. In such embodiments, electric circuit elements can form electrical connections between the DEP electrode regions 314 at the inner surface 242 of the flow region 240 and the second electrode 310 that can be selectively activated by the respective light patterns 322. When not activated, the electrical impedance through each electrical connection (i.e., from a corresponding DEP electrode region 314 on the inner surface 242, through the electrical connection, to the second electrode 310) can be greater than the impedance through the medium 202 (i.e., from the first electrode 304, through the medium 202, to the corresponding DEP electrode region 314 on the inner surface 242). When activated by light in the light pattern 322, however, the electrical impedance though the illuminated electrical connections (i.e., from each illuminated DEP electrode region 314a, through the electrical connection, to the second electrode 310) can be reduced to an amount less than the electrical impedance through the medium 202 (i.e., from the first electrode 304, through the medium 202, to the corresponding illuminated DEP electrode region 314a), thereby activating a DEP electrode at the corresponding DEP electrode region 314 as discussed above. DEP electrodes that attract or repel micro-objects (not shown) in the medium 202 can thus be selectively activated and deactivated at many different DEP electrode regions 314 at the inner surface 242 of the flow region 240 by the light pattern 322. Non-limiting examples of such configurations of the electrode activation substrate 308 include the phototransistor-based device 300 illustrated in FIGS. 21 and 22 of U.S. Pat. No. 7,956,339.

In other embodiments, the electrode activation substrate 308 can comprise a substrate comprising a plurality of electrodes, which may be photo-actuated. Non-limiting examples of such configurations of the electrode activation substrate 308 include the photo-actuated devices 200, 400, 500, and 600 illustrated and described in U.S. Patent Application Publication No. 2014/0124370. In still other embodiments, a DEP configuration of the support structure 104 and/or cover 122 does not rely upon light activation of DEP electrodes at the inner surface of the microfluidic device, but uses selectively addressable and energizable electrodes positioned opposite to a surface including at least one electrode, such as described in U.S. Pat. No. 6,942,776.

In some embodiments of a DEP configured device, the first electrode 304 can be part of a first wall 302 (or cover) of the housing 102, and the electrode activation substrate 308 and second electrode 310 can be part of a second wall 306 (or base) of the housing 102, generally as illustrated in FIG. 1D. As shown, the flow region 240 can be between the first wall 302 and the second wall 306. The foregoing, however, is but an example. In alternative embodiments, the first electrode 304 can be part of the second wall 306 and one or both of the electrode activation substrate 308 and/or the second electrode 310 can be part of the first wall 302. Moreover, the light source 320 can alternatively be located underneath the housing 102. In certain embodiments, the first electrode 304 may be an indium-tin-oxide (ITO) electrode, though other materials may also be used.

When used with the optically-actuated DEP configurations of microfluidic device 300 of FIGS. 1D-1E, a selector control module can thus select a micro-object (not shown) in the medium 202 in the flow region 240 by projecting one or more consecutive light patterns 322 into the device 300 to activate a corresponding one or more DEP electrodes at DEP electrode regions 314 of the inner surface 242 of the flow region 240 in successive patterns that surround and “capture” the micro-object. The selector control module can then move the captured micro-object within the flow region 240 by moving the light pattern 322 relative to the device 300 (or the device 300 (and thus the captured micro-object therein) can be moved relative to the light source 320 and/or light pattern 322). For embodiments featuring electrically-actuated DEP configurations of microfluidic device 300, the selector control module can select a micro-object (not shown) in the medium 202 in the flow region 240 by electrically activating a subset of DEP electrodes at DEP electrode regions 314 of the inner surface 242 of the flow region 240 that form a pattern that surrounds and “captures” the micro-object. The selector control module can then move the captured micro-object within the flow region 240 by changing the subset of DEP electrodes that are being electrically activated.

Growth chamber configurations. Non-limiting examples of growth chambers 136, 138, and 140 of device 100 are shown in FIGS. 1A-1C. With specific reference to FIG. 1C, each growth chamber 136, 138, 140 comprises an isolation structure 146 defining an isolation region 144 and a connection region 142 that fluidically connects the isolation region 144 to the flow channel 134. The connection regions 142 each have a proximal opening 152 into the flow channel 134, and a distal opening 154 into the respective isolation region 144. The connection regions 142 are preferably configured so that a maximum penetration depth of a flow of a fluidic medium (not shown) flowing at a maximum velocity (Vₘₐₓ) in the flow channel 134 does not inadvertently extend into the isolation region 144. A micro-object (not shown) or other material (not shown) disposed in an isolation region 144 of a respective growth chamber 136, 138, 140 can thus be isolated from, and not substantially affected by, a flow of medium (not shown) in the flow channel 134. The flow channel 134 can thus be an example of a swept region, and the isolation regions of the growth chambers 136, 138, 140 can be examples of unswept regions. As noted above, the respective flow channel 134 and growth chambers 136, 138, 140 are configured to contain one or more fluidic media (not shown). In the embodiment shown in FIGS. 1A-1C, the fluid access ports 124 are fluidically connected to the flow channel 134 and allow a fluidic medium (not shown) to be introduced into or removed from the microfluidic circuit 132. Once the microfluidic circuit 132 contains a fluidic medium, flows of specific fluidic media therein can be selectively generated in the flow channel 134. For example, a flow of a medium can be created from one fluid access port 124 functioning as an inlet to another fluid access port 124 functioning as an outlet.

FIG. 2 illustrates a detailed view of an example of a growth chamber 136 of the device 100 of FIGS. 1A-1C. Growth chambers 138, 140 can be configured similarly. Examples of micro-objects 222 located in growth chamber 136 are also shown.

As is known, a flow of fluidic medium 202 (indicated by directional arrow 212) in the microfluidic flow channel 134 past a proximal opening 152 of the growth chamber 136 can cause a secondary flow of the medium 202 (indicated by directional arrow 214) into and/or out of the growth chamber 136. To isolate the micro-objects 222 in the isolation region 144 of the growth chamber 136 from the secondary flow 214, the length L_{con} of the connection region 142 from the proximal opening 152 to the distal opening 154 is preferably greater than a maximum penetration depth Dₚ of the secondary flow 214 into the connection region 142 when the velocity of the flow 212 in the flow channel 134 is at a maximum (Vₘₐₓ). As long as the flow 212 in the flow channel 134 does not exceed the maximum velocity Vₘₐₓ, the flow 212 and resulting secondary flow 214 are limited to the respective flow channel 134 and connection region 142, and kept out of the isolation region 144 of the growth chamber 136. The flow 212 in the flow channel 134 will thus not draw micro-objects 222 out of the isolation region 144 of growth chamber 136.

Moreover, the flow 212 will not move miscellaneous particles (e.g., microparticles and/or nanoparticles) that may be located in the flow channel 134 into the isolation region 144 of the growth chamber 136. Having the length L_{con} of the connection region 142 be greater than the maximum penetration depth Dₚ can thus prevent contamination of the growth chamber 136 with miscellaneous particles from the flow channel 134 or from another growth chamber 138, 140.

Because the flow channel 134 and the connection regions 142 of the growth chambers 136, 138, 140 can be affected by the flow 212 of medium 202 in the flow channel 134, the flow channel 134 and connection regions 142 can be deemed swept (or flow) regions of the microfluidic circuit 132. The isolation regions 144 of the growth chambers 136, 138, 140, on the other hand, can be deemed unswept (or non-flow) regions. For example, components (not shown) in a first medium 202 in the flow channel 134 can mix with a second medium 204 in the isolation region 144 substantially only by diffusion of the components of the first medium 202 from the flow channel 134 through the connection region 142 and into the second medium 204 in the isolation region 144. Similarly, components of the second medium 204 (not shown) in the isolation region 144 can mix with the first medium 202 in the flow channel 134 substantially only by diffusion of the components of the second medium 204 from the isolation region 144 through the connection region 142 and into the first medium 202 in the flow channel 134. It should be appreciated that the first medium 202 can be the same medium or a different medium than the second medium 204. Moreover, the first medium 202 and the second medium 204 can start out being the same, then become different, e.g., through conditioning of the second medium by one or more cells in the isolation region 144, or by changing the medium flowing through the flow channel 134.

The maximum penetration depth Dₚ of the secondary flow 214 caused by the flow 212 in the flow channel 134 can depend on a number of parameters. Examples of such parameters include (without limitation) the shape of the flow channel 134 (e.g., the channel can direct medium into the connection region 142, divert medium away from the connection region 142, or simply flow past the connection region 142); a width W_{ch} (or cross-sectional area) of the flow channel 134 at the proximal opening 152; a width W_{con} (or cross-sectional area) of the connection region 142 at the proximal opening 152; the maximum velocity Vₘₐₓ of the flow 212 in the flow channel 134; the viscosity of the first medium 202 and/or the second medium 204, and the like.

In some embodiments, the dimensions of the flow channel 134 and/or growth chambers 136, 138, 140 are oriented as follows with respect to the flow 212 in the flow channel 134: the flow channel width W_{ch} (or cross-sectional area of the flow channel 134) can be substantially perpendicular to the flow 212; the width W_{con} (or cross-sectional area) of the connection region 142 at the proximal opening 152 can be substantially parallel to the flow 212; and the length L_{con} of the connection region can be substantially perpendicular to the flow 212. The foregoing are examples only, and the dimensions of the flow channel 134 and growth chambers 136, 138, 140 can be in additional and/or further orientations with respect to each other.

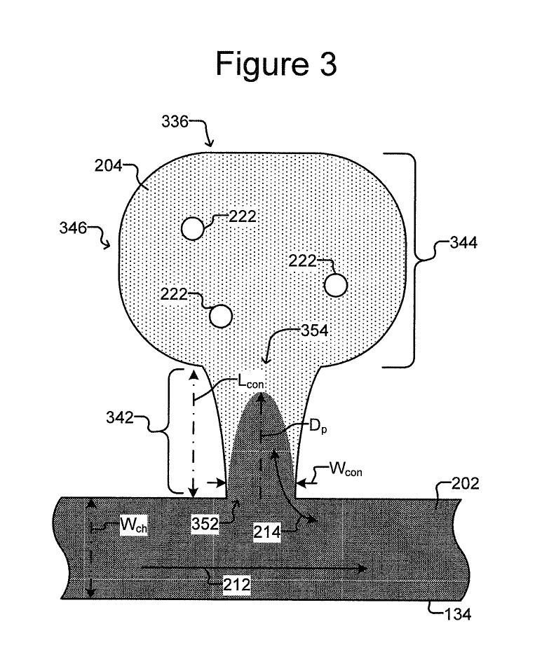

As illustrated in FIG. 2, the width W_{con} of the connection region 142 can be uniform from the proximal opening 152 to the distal opening 154. The width W_{con} of the connection region 142 at the distal opening 154 can thus be in any of the below-identified ranges corresponding to the width W_{con} of the connection region 142 at the proximal opening 152. Alternatively, the width W_{con} of the connection region 142 at the distal opening 154 can be larger (e.g., as shown in the embodiment of FIG. 3) or smaller (e.g., as shown in the embodiment of FIGS. 4A-4C) than the width W_{con} of the connection region 142 at the proximal opening 152.

As also illustrated in FIG. 2, the width of the isolation region 144 at the distal opening 154 can be substantially the same as the width W_{con} of the connection region 142 at the proximal opening 152. The width of the isolation region 144 at the distal opening 154 can thus be in any of the below-identified ranges corresponding to the width W_{con} of the connection region 142 at the proximal opening 152. Alternatively, the width of the isolation region 144 at the distal opening 154 can be larger (e.g., as shown in FIG. 3) or smaller (not shown) than the width W_{con} of the connection region 142 at the proximal opening 152.

In some embodiments, the maximum velocity Vₘₐₓ of a flow 212 in the flow channel 134 is substantially the same as the maximum velocity that the flow channel 134 can maintain without causing a structural failure in the respective microfluidic device (e.g., device 100) in which the flow channel is located. In general, the maximum velocity that a flow channel can maintain depends on various factors, including the structural integrity of the microfluidic device and the cross-sectional area of the flow channel. For the exemplary microfluidic devices disclosed and described herein, a maximum flow velocity Vₘₐₓ in a flow channel having a cross-sectional area of about 3,500 to 10,000 square microns, is about 1.5 to 15 microliters/sec. Alternatively, the maximum velocity Vₘₐₓ of a flow in a flow channel can be set so as to ensure that isolation regions are isolated from the flow in the flow channel. In particular, based on the width W_{con} of the proximal opening of a connection region of a growth chamber, Vₘₐₓ can be set so as to ensure that the depth of penetration Dₚ of a secondary flow into the connection region is less than L_{con}. For example, for a growth chamber having a connection region with a proximal opening having a width W_{con} of W about 40 to 50 microns and L_{con} of about 50 to 100 microns, Vₘₐₓ can be set at or about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 microliters/sec.

In some embodiments, the sum of the length L_{con} of the connection region 142 and a corresponding length of the isolation region 144 of a growth chamber 136, 138, 140 can be sufficiently short for relatively rapid diffusion of components of a second medium 204 contained in the isolation region 144 to a first medium 202 flowing or otherwise contained in the flow channel 134. For example, in some embodiments, the sum of (1) the length L_{con} of the connection region 142 and (2) the distance between a biological micro-object located in isolation region 144 of a growth chamber 136, 138, 140 and the distal opening 154 of the connection region can be one of the following ranges: from about 40 microns to 500 microns, 50 microns to 450 microns, 60 microns to 400 microns, 70 microns to 350 microns, 80 microns to 300 microns, 90 microns to 250 microns, 100 microns to 200 microns, or any range including one of the foregoing end points. The rate of diffusion of a molecule (e.g., an analyte of interest, such as an antibody) is dependent on a number of factors, including (without limitation) temperature, viscosity of the medium, and the coefficient of diffusion D₀ of the molecule. For example, the D₀ for an IgG antibody in aqueous solution at about 20° C. is about 4.4×10⁻⁷ cm²/sec, while the kinematic viscosity of cell culturing medium is about 9×10⁻⁴ m²/sec. Thus, an antibody in cell culturing medium at about 20° C. can have a rate of diffusion of about 0.5 microns/sec. Accordingly, in some embodiments, a time period for diffusion from a biological micro-object located in isolation region 144 into the flow channel 134 can be about 10 minutes or less (e.g., about 9, 8, 7, 6, 5 minutes, or less). The time period for diffusion can be manipulated by changing parameters that influence the rate of diffusion. For example, the temperature of the media can be increased (e.g., to a physiological temperature such as about 37° C.) or decreased (e.g., to about 15° C., 10° C., or 4° C.) thereby increasing or decreasing the rate of diffusion, respectively. Alternatively, or in addition, the concentrations of solutes in the medium can be increased or decreased.

The physical configuration of the growth chamber 136 illustrated in FIG. 2 is but an example, and many other configurations and variations for growth chambers are possible. For example, the isolation region 144 is illustrated as sized to contain a plurality of micro-objects 222, but the isolation region 144 can be sized to contain only about one, two, three, four, five, or similar relatively small numbers of micro-objects 222. Accordingly, the volume of an isolation region 144 can be, for example, at least about 3×10³, 6×10³, 9×10³, 1×10⁴, 2×10⁴, 4×10⁴, 8×10⁴, 1×10⁵, 2×10⁵, 4×10⁵, 8×10⁵, 1×10⁶, 2×10⁶, 4×10⁶, 6×10⁶ cubic microns, or more.

As another example, the growth chamber 136 is shown in FIG. 2 as extending generally perpendicularly from the flow channel 134 and thus forming generally about 90° angles with the flow channel 134. The growth chamber 136 can alternatively extend from the flow channel 134 at other angles such as, for example, any angle from about 30° to about 150°.

As yet another example, the connection region 142 and the isolation region 144 are illustrated in FIG. 2 as having a substantially rectangular configuration, but one or both of the connection region 142 and the isolation region 144 can have a different configuration, including (without limitation) oval, triangular, circular, hourglass-shaped, and the like.

As still another example, the connection region 142 and the isolation region 144 are illustrated in FIG. 2 as having substantially uniform widths. That is, the width W_{con} of the connection region 142 is shown as being uniform along the entire length L_{con} from the proximal opening 152 to the distal opening 154. A corresponding width of the isolation region 144 is similarly uniform; and the width W_{con} of the connection region 142 and a corresponding width of the isolation region 144 are shown as equal. However, in alternate embodiments, any of the foregoing can be different. For example, a width W_{con} of the connection region 142 can vary along the length L_{con}, from the proximal opening 152 to the distal opening 154, e.g., in the manner of a trapezoid, or of an hourglass; a width of the isolation region 144 can also vary along the length L_{con}, e.g., in the manner of a triangle, or of a flask; and a width W_{con} of the connection region 142 can be different than a width of the isolation region 144.

FIG. 3 illustrates an alternate embodiment of a growth chamber 336, demonstrating some examples of the foregoing variations. While the alternative growth chamber 336 is described as a replacement for chamber 136 in the microfluidic device 100, it should be appreciated that the growth chamber 336 can replace any of growth chambers in any of the microfluidic device embodiments disclosed or described herein. Furthermore, there may be one growth chamber 336 or a plurality of growth chambers 336 provided in a given microfluidic device.

The growth chamber 336 includes a connection region 342 and an isolation structure 346 comprising an isolation region 344. The connection region 342 has a proximal opening 352 to the flow channel 134 and a distal opening 354 to the isolation region 344. In the embodiment illustrated in FIG. 3, the connection region 342 expands such that its width W_{con} increases along a length of the connection region L_{con}, from the proximal opening 352 to the distal opening 354. Other than having a different shape, however, the connection region 342, isolation structure 346, and isolation region 344 function generally the same as the above-described connection region 142, isolation structure 146, and isolation region 144 of growth chamber 136 shown in FIG. 2.

For example, the flow channel 134 and the growth chamber 336 can be configured so that the maximum penetration depth Dₚ of the secondary flow 214 extends into the connection region 342, but not into the isolation region 344. The length L_{con} of the connection region 342 can thus be greater than the maximum penetration depth Dₚ, generally as discussed above with respect to the connection regions 142 shown in FIG. 2. Also, as discussed above, micro-objects 222 in the isolation region 344 will stay in the isolation region 344 as long as the velocity of the flow 212 in the flow channel 134 does not exceed the maximum flow velocity Vₘₐₓ. The flow channel 134 and connection region 342 are thus examples of swept (or flow) regions, and the isolation region 344 is an example of an unswept (or non-flow) region.

FIGS. 4A-C depict another exemplary embodiment of a microfluidic device 400 containing a microfluidic circuit 432 and flow channels 434, which are variations of the respective microfluidic device 100, circuit 132 and flow channel 134 of FIGS. 1A-1C. The microfluidic device 400 also has a plurality of growth chambers 436 that are additional variations of the above-described growth chambers 136, 138, 140 and 336. In particular, it should be appreciated that the growth chambers 436 of device 400 shown in FIGS. 4A-C can replace any of the above-described growth chambers 136, 138, 140, 336 in devices 100 and 300. Likewise, the microfluidic device 400 is another variant of the microfluidic device 100, and may also have the same or a different DEP configuration as the above-described microfluidic device 300, as well as any of the other microfluidic system components described herein.

The microfluidic device 400 of FIGS. 4A-C comprises a support structure (not visible in FIGS. 4A-C, but can be the same or generally similar to the support structure 104 of device 100 depicted in FIGS. 1A-1C), a microfluidic circuit structure 412, and a cover (not visible in FIGS. 4A-C, but can be the same or generally similar to the cover 122 of device 100 depicted in FIGS. 1A-1C). The microfluidic circuit structure 412 includes a frame 414 and microfluidic circuit material 416, which can be the same as or generally similar to the frame 114 and microfluidic circuit material 116 of device 100 shown in FIGS. 1A-1C. As shown in FIG. 4A, the microfluidic circuit 432 defined by the microfluidic circuit material 416 can comprise multiple flow channels 434 (two are shown but there can be more) to which multiple growth chambers 436 are fluidically connected.

Each growth chamber 436 can comprise an isolation structure 446, an isolation region 444 within the isolation structure 446, and a connection region 442. From a proximal opening 472 at the flow channel 434 to a distal opening 474 at the isolation structure 436, the connection region 442 fluidically connects the flow channel 434 to the isolation region 444. Generally in accordance with the above discussion of FIG. 2, a flow 482 of a first fluidic medium 402 in a flow channel 434 can create secondary flows 484 of the first medium 402 from the flow channel 434 into and/or out of the respective connection regions 442 of the growth chambers 436.

As illustrated in FIG. 4B, the connection region 442 of each growth chamber 436 generally includes the area extending between the proximal opening 472 to a flow channel 434 and the distal opening 474 to an isolation structure 446. The length L_{con} of the connection region 442 can be greater than the maximum penetration depth Dₚ of secondary flow 484, in which case the secondary flow 484 will extend into the connection region 442 without being redirected toward the isolation region 444 (as shown in FIG. 4A). Alternatively, at illustrated in FIG. 4C, the connection region 442 can have a length L_{con} that is less than the maximum penetration depth Dₚ, in which case the secondary flow 484 will extend through the connection region 442 and be redirected toward the isolation region 444. In this latter situation, the sum of lengths L_{c1} and L_{c2} of connection region 442 is greater than the maximum penetration depth Dₚ, so that secondary flow 484 will not extend into isolation region 444. Whether length L_{con} of connection region 442 is greater than the penetration depth Dₚ, or the sum of lengths L_{c1} and L_{c2} of connection region 442 is greater than the penetration depth Dₚ, a flow 482 of a first medium 402 in flow channel 434 that does not exceed a maximum velocity Vₘₐₓ will produce a secondary flow having a penetration depth Dₚ, and micro-objects (not shown but can be the same or generally similar to the micro-objects 222 shown in FIG. 2) in the isolation region 444 of a growth chamber 436 will not be drawn out of the isolation region 444 by a flow 482 of first medium 402 in flow channel 434. Nor will the flow 482 in flow channel 434 draw miscellaneous materials (not shown) from flow channel 434 into the isolation region 444 of a growth chamber 436. As such, diffusion is the only mechanism by which components in a first medium 402 in the flow channel 434 can move from the flow channel 434 into a second medium 404 in an isolation region 444 of a growth chamber 436. Likewise, diffusion is the only mechanism by which components in a second medium 404 in an isolation region 444 of a growth chamber 436 can move from the isolation region 444 to a first medium 402 in the flow channel 434. The first medium 402 can be the same medium as the second medium 404, or the first medium 402 can be a different medium than the second medium 404. Alternatively, the first medium 402 and the second medium 404 can start out being the same, then become different, e.g., through conditioning of the second medium by one or more cells in the isolation region 444, or by changing the medium flowing through the flow channel 434.

As illustrated in FIG. 4B, the width W_{ch} of the flow channels 434 (i.e., taken transverse to the direction of a fluid medium flow through the flow channel indicated by arrows 482 in FIG. 4A) in the flow channel 434 can be substantially perpendicular to a width W_{con1} of the proximal opening 472 and thus substantially parallel to a width W_{con2} of the distal opening 474. The width W_{con1} of the proximal opening 472 and the width W_{con2} of the distal opening 474, however, need not be substantially perpendicular to each other. For example, an angle between an axis (not shown) on which the width W_{con1} of the proximal opening 472 is oriented and another axis on which the width W_{con2} of the distal opening 474 is oriented can be other than perpendicular and thus other than 90°. Examples of alternatively angles include angles in any of the following ranges: from about 30° to about 90°, from about 45° to about 90°, from about 60° to about 90°, or the like.

In various embodiments of growth chambers 136, 138, 140, 336, or 436, the isolation region of the growth chamber may have a volume configured to support no more than about 1×10³, 5×10², 4×10², 3×10², 2×10², 1×10², 50, 25, 15, or 10 cells in culture. In other embodiments, the isolation region of the growth chamber has a volume to support up to and including about 1×10³, 1×10⁴, or 1×10⁵ cells.

In various embodiments of growth chambers 136, 138, 140, 336, or 436, the width W_{ch} of the flow channel 134 at a proximal opening 152 (growth chambers 136, 138, or 14); the width W_{ch} of the flow channel 134 at a proximal opening 352 (growth chambers 336); or the width W_{ch} of the flow channel 434 at a proximal opening 472 (growth chambers 436) can be any of the following ranges: from about 50-1000 microns, 50-500 microns, 50-400 microns, 50-300 microns, 50-250 microns, 50-200 microns, 50-150 microns, 50-100 microns, 70-500 microns, 70-400 microns, 70-300 microns, 70-250 microns, 70-200 microns, 70-150 microns, 90-400 microns, 90-300 microns, 90-250 microns, 90-200 microns, 90-150 microns, 100-300 microns, 100-250 microns, 100-200 microns, 100-150 microns, and 100-120 microns. The foregoing are examples only, and the width W_{ch} of the flow channel 134 or 434 can be in other ranges (e.g., a range defined by any of the endpoints listed above).

In various embodiments of growth chambers 136, 138, 140, 336, or 436, the height H_{ch} of the flow channel 134 at a proximal opening 152 (growth chambers 136, 138, or 140), the flow channel 134 at a proximal opening 352 (growth chambers 336), or the flow channel 434 at a proximal opening 472 (growth chambers 436) can be any of the following ranges: from about 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{ch} of the flow channel 134 or 434 can be in other ranges (e.g., a range defined by any of the endpoints listed above).

In various embodiments of growth chambers 136, 138, 140, 336, or 436, a cross-sectional area of the flow channel 134 at a proximal opening 152 (growth chambers 136, 138, or 140), the flow channel 134 at a proximal opening 352 (growth chambers 336), or the flow channel 434 at a proximal opening 472 (growth chambers 436) can be any of the following ranges: from about 500-50,000 square microns, 500-40,000 square microns, 500-30,000 square microns, 500-25,000 square microns, 500-20,000 square microns, 500-15,000 square microns, 500-10,000 square microns, 500-7,500 square microns, 500-5,000 square microns, 1,000-25,000 square microns, 1,000-20,000 square microns, 1,000-15,000 square microns, 1,000-10,000 square microns, 1,000-7,500 square microns, 1,000-5,000 square microns, 2,000-20,000 square microns, 2,000-15,000 square microns, 2,000-10,000 square microns, 2,000-7,500 square microns, 2,000-6,000 square microns, 3,000-20,000 square microns, 3,000-15,000 square microns, 3,000-10,000 square microns, 3,000-7,500 square microns, or 3,000 to 6,000 square microns. The foregoing are examples only, and the cross-sectional area of the flow channel 134 at a proximal opening 152, the flow channel 134 at a proximal opening 352, or the flow channel 434 at a proximal opening 472 can be in other ranges (e.g., a range defined by any of the endpoints listed above).

In various embodiments of growth chambers 136, 138, 140, 336, or 436, the length of the connection region L_{con} can be any of the following ranges: from about 1-200 microns, 5-150 microns, 10-100 microns, 15-80 microns, 20-60 microns, 20-500 microns, 40-400 microns, 60-300 microns, 80-200 microns, and 100-150 microns. The foregoing are examples only, and length L_{con} of a connection region 142 (growth chambers 136, 138, or 140), connection region 342 (growth chambers 336), or connection region 442 (growth chambers 436) can be in a different ranges than the foregoing examples (e.g., a range defined by any of the endpoints listed above).

In various embodiments of growth chambers 136, 138, 140, 336, or 436, the width W_{con} of a connection region 142 at a proximal opening 152 (growth chambers 136, 138, or 140, connection region 342 at a proximal opening 352 (growth chambers 336), or a connection region 442 at a proximal opening 472 (growth chambers 436) can be any of the following ranges: from about 20-500 microns, 20-400 microns, 20-300 microns, 20-200 microns, 20-150 microns, 20-100 microns, 20-80 microns, 20-60 microns, 30-400 microns, 30-300 microns, 30-200 microns, 30-150 microns, 30-100 microns, 30-80 microns, 30-60 microns, 40-300 microns, 40-200 microns, 40-150 microns, 40-100 microns, 40-80 microns, 40-60 microns, 50-250 microns, 50-200 microns, 50-150 microns, 50-100 microns, 50-80 microns, 60-200 microns, 60-150 microns, 60-100 microns, 60-80 microns, 70-150 microns, 70-100 microns, and 80-100 microns. The foregoing are examples only, and the width W_{con} of a connection region 142 at a proximal opening 152; connection region 342 at a proximal opening 352; or a connection region 442 at a proximal opening 472 can be different than the foregoing examples (e.g., a range defined by any of the endpoints listed above).

In various embodiments of growth chambers 136, 138, 140, 336, or 436, the width W_{con} of a connection region 142 at a proximal opening 152 (growth chambers 136, 138, or 140), a connection region 342 at a proximal opening 352 (growth chambers 336), or a connection region 442 at a proximal opening 472 (growth chambers 436) can be any of the following ranges: from about 2-35 microns, 2-25 microns, 2-20 microns, 2-15 microns, 2-10 microns, 2-7 microns, 2-5 microns, 2-3 microns, 3-25 microns, 3-20 microns, 3-15 microns, 3-10 microns, 3-7 microns, 3-5 microns, 3-4 microns, 4-20 microns, 4-15 microns, 4-10 microns, 4-7 microns, 4-5 microns, 5-15 microns, 5-10 microns, 5-7 microns, 6-15 microns, 6-10 microns, 6-7 microns, 7-15 microns, 7-10 microns, 8-15 microns, and 8-10 microns. The foregoing are examples only, and the width W_{con} of a connection region 142 at a proximal opening 152, a connection region 342 at a proximal opening 352, or a connection region 442 at a proximal opening 472 can be different than the foregoing examples (e.g., a range defined by any of the endpoints listed above).

In various embodiments of growth chambers 136, 138, 140, 336, or 436, a ratio of the length L_{con} of a connection region 142 to a width W of the connection region 142 at the proximal opening 152 (growth chambers 136, 138; 140), a ratio of the length L_{con} of a connection region 342 to a width W_{con} of the connection region 342 at the proximal opening 352 (growth chambers 336), or a ratio of the length L_{con} of a connection region 442 to a width W_{con} of the connection region a connection region 442 to a width W of the connection region 442 at the proximal opening 472 (growth chambers 436) can be greater than or equal to any of the following ratios: about 0.5, 1.0, 1.5, 2.0, 2.5, 3.0, 3.5, 4.0, 4.5, 5.0, 6.0, 7.0, 8.0, 9.0, 10.0, or more. The foregoing are examples only, and the ratio of the length L_{con} of a connection region 142 to a width W_{con} of the connection region 142 at the proximal opening 152, the ratio of the length L_{con} of a connection region 342 to a width W_{con} the connection region 342 at the proximal opening 372; or the ratio of the length L_{con} of a connection region 442 to a width W_{con} of the connection region 442 at the proximal opening 472 can be different than the foregoing examples.

In various embodiments of microfluidic devices having growth chambers 136, 138, 140, 336, or 436, Vₘₐₓ can be set at about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 microliters/sec, or higher (e.g., about 3.0, 4.0, 5.0 microliters/sec, or more).

In various embodiments of microfluidic devices having growth chambers 136, 138, 140, 336, or 436, the volume of an isolation region 144 (growth chambers 136, 138, or 140), 344 (growth chambers 336) or 444 (growth chambers 436) can be, for example, at least about 3×10³, 6×10³, 9×10³, 1×10⁴, 2×10⁴, 4×10⁴, 8×10⁴, 1×10⁵, 2×10⁵, 4×10⁵, 8×10⁵, 1×10⁶, 2×10⁶, 4×10⁶, 6×10⁶ cubic microns, or more.

In some embodiments, the microfluidic device has growth chambers 136, 138, 140, 336, or 436, wherein no more than about 1×10² biological cells may be maintained, and the volume of the growth chambers may be no more than about 2×10⁶ cubic microns.

In some embodiments, the microfluidic device has growth chambers 136, 138, 140, 336, or 436, wherein no more than about 1×10² biological cells may be maintained, and the volume of the growth chambers may be no more than about 4×10⁵ cubic microns.

In yet other embodiments, the microfluidic device has growth chambers 136, 138, 140, 336, or 436, wherein no more than about 50 biological cells may be maintained, and the volume of the growth chambers may be no more than about 4×10⁵ cubic microns.

In various embodiment, the microfluidic device has growth chambers configured as in any of the embodiments discussed herein where the microfluidic device has about 100 to about 500 growth chambers; about 200 to about 1000 growth chambers, about 500 to about 1500 growth chambers, about 1000 to about 2000 growth chambers, or about 1000 to about 3500 growth chambers.

In some other embodiments, the microfluidic device has growth chambers configured as in any of the embodiments discussed herein where the microfluidic device has about 1500 to about 3000 growth chambers, about 2000 to about 3500 growth chambers, about 2000 to about 4000 growth chambers, about 2500 to about 4000 growth chambers, or about 3000 to about 4500 growth chambers.

In some embodiments, the microfluidic device has growth chambers configured as in any of the embodiments discussed herein where the microfluidic device has about 3000 to about 4500 growth chambers, about 3500 to about 5000 growth chambers, about 4000 to about 5500 chambers, about 4500 to about 6000 growth chambers or about 5000 to about 6500 chambers.

In further embodiments, the microfluidic device has growth chambers configured as in any of the embodiments discussed herein, where the microfluidic device has about 6000 to about 7500 growth chambers, about 7000 to about 8500 growth chambers, about 8000 to about 9500 growth chambers, about 9000 to about 10,500 growth chambers, about, about 10, 000 to about 11,500 growth chambers, about 11,000 to about 12,500 growth chambers, about 12,000 to about 13,500 growth chambers, about 13,000 to about 14,500 growth chambers about 14,000 to about 15,500 growth chambers, about 15,000 to about 16,500 growth chambers, about 16,000 to about 17,500 growth chambers, about 17,000 to about 18,500 growth chambers.

In various embodiments, the microfluidic device has growth chambers configured as in any of the embodiments discussed herein, where the microfluidic device has about 18,000 to about 19,500 growth chambers, about 18,500 to about 20,000 growth chambers, about 19,000 to about 20,500 growth chambers, about 19,500 to about 21,000 growth chambers, or about 20,000 to about 21,500 growth chambers.

Other properties of the growth chambers. Although the barriers of microfluidic circuit material 116 (FIGS. 1A-1C) and 416 (FIGS. 4A-4C) that define the respective growth chambers 136, 138, 140 of device 100 (FIGS. 1A-1C) and form the isolation structure 446 of growth chambers 436 of device 400 (FIGS. 4A-4C) are illustrated and discussed above as physical barriers, it should be appreciated that the barriers can alternatively be created as “virtual” barriers comprising DEP forces activated by light in the light pattern 322.

In some other embodiments, respective growth chambers 136, 138, 140, 336 and 436 can be shielded from illumination (e.g., by the detector and/or the selector control module directing the light source 320), or can be only selectively illuminated for brief periods of time. Cells and other biological micro-objects contained in the growth chambers can thus be protected from further (i.e., possibly hazardous) illumination after being moved into the growth chambers 136, 138, 140, 336 and 436.

Fluidic medium. With regard to the foregoing discussion about microfluidic devices having a flow channel and one or more growth chambers, a fluidic medium (e.g., a first medium and/or a second medium) can be any fluid that is capable of maintaining a biological micro-object in a substantially assayable state. The assayable state will depend on the biological micro-object and the assay being performed. For example, if the biological micro-object is a cell that is being assayed for the secretion of a protein of interest, the cell would be substantially assayable provided that the cell is viable and capable of expressing and secreting proteins. Alternatively, the fluidic medium can be any fluid that is capable of expanding the cells or maintaining the cells in a state such that they are still capable of expanding (i.e., increasing in number due to mitotic cell division). Many different types of fluidic medium, particularly cell culturing medium, are known in the art, and what is a suitable medium will typically depend on the types of cells being cultured. In certain embodiments, the cell culturing medium will include mammalian serum, such as fetal bovine serum (FBS) or calf serum. In other embodiments, the cell culturing medium may be serum free. In either case, the cell culturing medium may be supplemented with various nutrients, such as vitamins, minerals, and/or antibiotics.

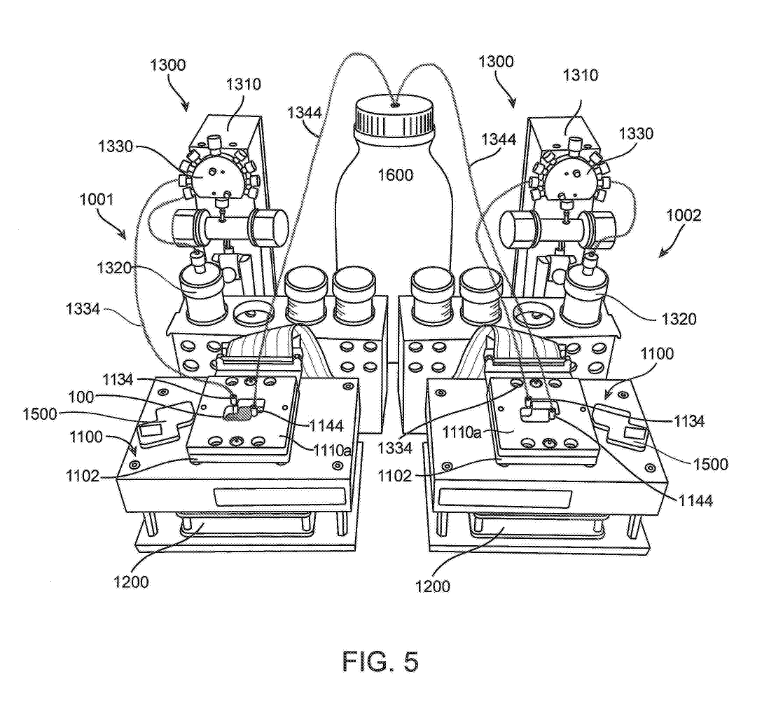

Culturing Station. FIG. 5 depicts a pair of exemplary culturing stations, 1001 and 1002, disposed in a side-by-side configuration to be used for culturing biological cells in the above-described microfluidic devices (e.g., device 100 of FIGS. 1A-1C). For ease in illustration and disclosure, features, components and configurations of the culturing stations 1001/1002 are given the same reference numbers as the corresponding features, components and configurations disclosed or described in other sections of this document. Each culturing station 1001/1002 includes a thermally regulated mounting interface 1100 configured to have a microfluidic device 100 detachably mounted thereon. For purposes of illustration, the device mounting interface 1100 of the culturing station 1001 has a microfluidic device 100 mounted thereon; whereas the device mounting interface 1100 of the culturing station 1002 does not. Each culturing station 1001/1002 includes a thermal regulation system 1200 (shown in part) configured for precisely controlling a temperature of a microfluidic device 100 detachably mounted on a mounting interface 1100 of the respective culturing station 1001/1002. Each culturing station 1001/1002 further includes a media perfusion system 1300 configured to controllably and selectively dispense a flowable culturing media into a microfluidic device 100 securely mounted on the corresponding mounting interface 1100.

Each media perfusion system 1300 includes a pump 1310 having an input fluidically connected to a source of culturing media 1320 and a multi-position valve 1330 that selectively and fluidically connects an output of the pump 1310 with a perfusion line 1334. The perfusion line 1334 is associated with a respective mounting interface 1100 and configured to be fluidically connected to a fluid ingress port 124 of a microfluidic device 100 mounted on the respective mounting interface 1100 (the ingress port 124 on the microfluidic device 100 shown in FIG. 5 is obscured by the below-described microfluidic device cover). A control system (not shown) is configured to selectively operate the pump 1310 and multi-position valve 1330 to thereby selectively cause culturing medium from the culturing media source 1320 to flow through the perfusion line 1334 at a controlled flow rate for a controlled period of time. More particularly, the control system is preferably programmed or may be programmed through operator input to provide an intermittent flow of the culturing medium through the perfusion line 1334 according to an on-off duty cycle and a flow rate, as discussed further below. The on-off duty cycle and/or flow rate may be based at least in part on input received through a user interface (not shown).

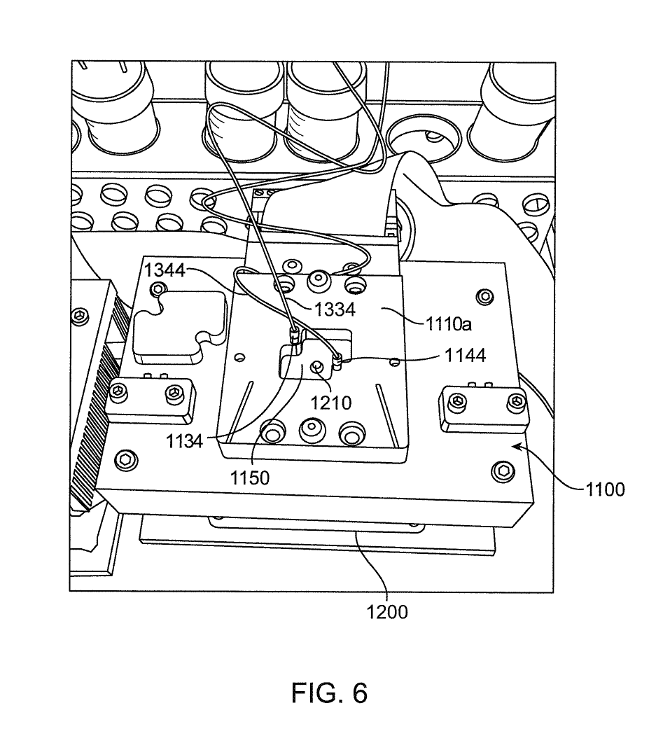

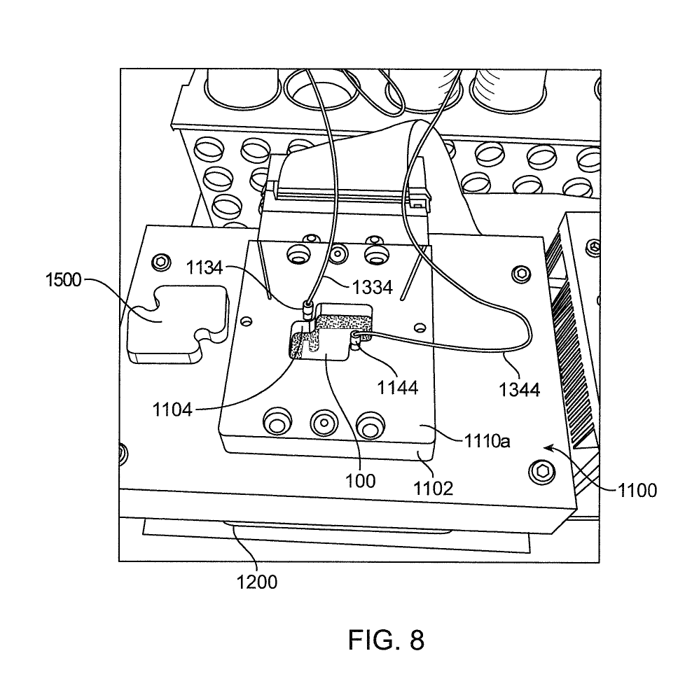

With additional reference to FIG. 6, the microfluidic device mounting interface 1100 can include a microfluidic device cover 1110 (1110a in FIG. 6) configured to at least partially enclose a microfluidic device mounted on the mounting interface 1100. By enclosing the microfluidic device on the mounting interface, the microfluidic device cover 1110 can facilitate the proper positioning of the microfluidic device on the mounting interface 1100 and/or ensure that the microfluidic device is securely held against the mounting interface 1100. The microfluidic device covers 1110a shown in FIGS. 5, 6, and 8 are secured (each by a respective pair of screws) to their respective mounting interfaces 1100. In FIGS. 5 and 8, the microfluidic device cover 1110a of the mounting interface 1100 of the culturing station 1001 encloses a microfluidic device 100. As shown, a distal end connector 1134 can be coupled to the microfluidic device cover 1110a and configured, along with the microfluidic device cover 1110a, to receive and fluidically connect the perfusion line 1334 to the fluid ingress port 124 of a mounted microfluidic device 100 enclosed (e.g., properly positioned and securely held) by the microfluidic device cover 1110a. By way of example, the microfluidic device cover 1110a and/or distal end connector 1134 may include one or more features configured to form a pressure fit, a frictional fit, or another type of fluid tight connection between the distal end of the perfusion line 1334 and the respective fluid ingress port 124 of the microfluidic device 100, in order to fluidically connect the perfusion line 1334 to the microfluidic circuit 134 of the device 100.

A waste line 1344 can also be associated with the mounting interface 1100. For example, as shown in FIGS. 5 and 6, a waste line 1344 can be connected to the microfluidic device cover 1110a via a proximal end connector 1144 coupled to the microfluidic device cover 1110a. The proximal end connector 1144 can be configured, in conjunction with a configuration of the microfluidic device cover 1110a, so that the proximal end of the waste line 1344 is fluidically connected to a fluid egress port 124 (obscured by the microfluidic device cover 1110a in FIG. 5) on the microfluidic device 100 when the microfluidic device 100 is enclosed (e.g., properly positioned and securely held) by the microfluidic device cover 1110a. By way of example, each microfluidic device cover 1110a may include one or more features configured to form a pressure fit, a frictional fit, or another type of fluid tight connection between the proximal end of a waste line 1344 and the fluid egress port 124 of the microfluidic device 100, in order to fluidically connect the waste line 1344 to the microfluidic circuit 134 of the microfluidic device 100. The distal end of the waste line 1344 can be connected and/or fluidically coupled to a waste container 1600. As depicted in FIG. 5, the culturing stations 1001 and 1002 share a common waste container 1600. However, it should be appreciated that each culturing station 1001/1002 may have its own waste container 1600.

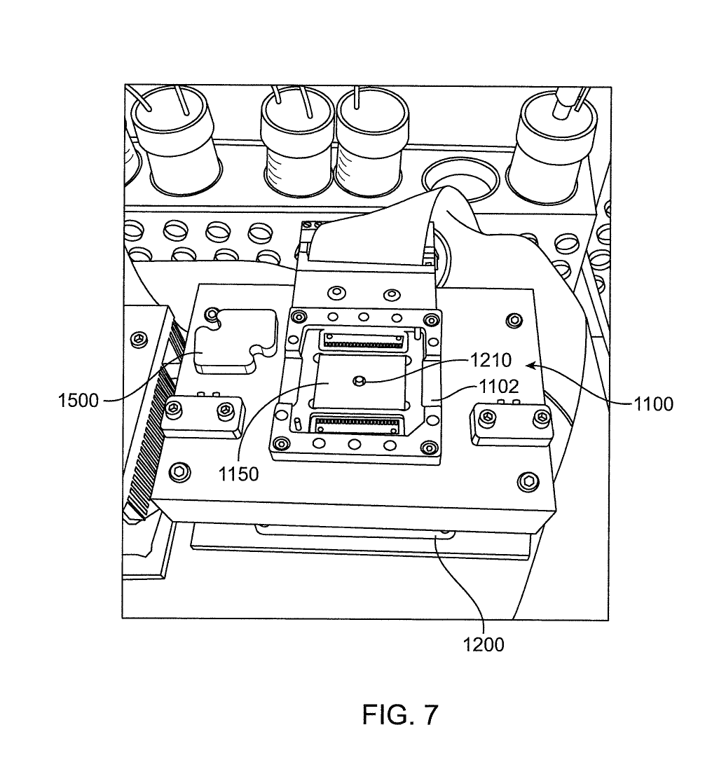

With additional reference to FIG. 7, a mounting interface 1100 can comprise a metallic substrate 1150, which may comprise a generally planartop surface configured to thermally couple with a generally planar metallic bottom surface (not shown) of a microfluidic device 100 mounted thereon. A frame 1102 can be attached or positioned proximal to the surface of the substrate 1150 to define a mounting area for the microfluidic device 100. The metallic substrate 1150 can comprise a metal having a high degree of thermal conductivity, such as copper. In a particular embodiment, the metal can be a copper alloy, such as brass or bronze.

As best seen in FIG. 8, the microfluidic device cover 1110a can include a window 1104 to allow for imaging of the microfluidic device 100 mounted on the mounting interface substrate 1150 (within the frame 1102 in FIG. 7) and securely enclosed by the microfluidic device cover 1110a. As shown in FIGS. 5-8, the mounting interface 1100 can further include a lid 1500 that may be disposed upon the microfluidic device cover 1110a (e.g., over the window 1104) of the mounting interface 1100 when imaging of the microfluidic device 100 through the window 1104 of the microfluidic device cover 1110a is not taking place. As shown, the lid 1500 can be shaped and sized to substantially prevent light from passing directly through the window 1104 of the microfluidic device cover 1110a and into the microfluidic device 100. To further reduce the amount of light incident upon the surface of the microfluidic device 100, the lid 1500 can be composed of an opaque and/or light-reflecting material.

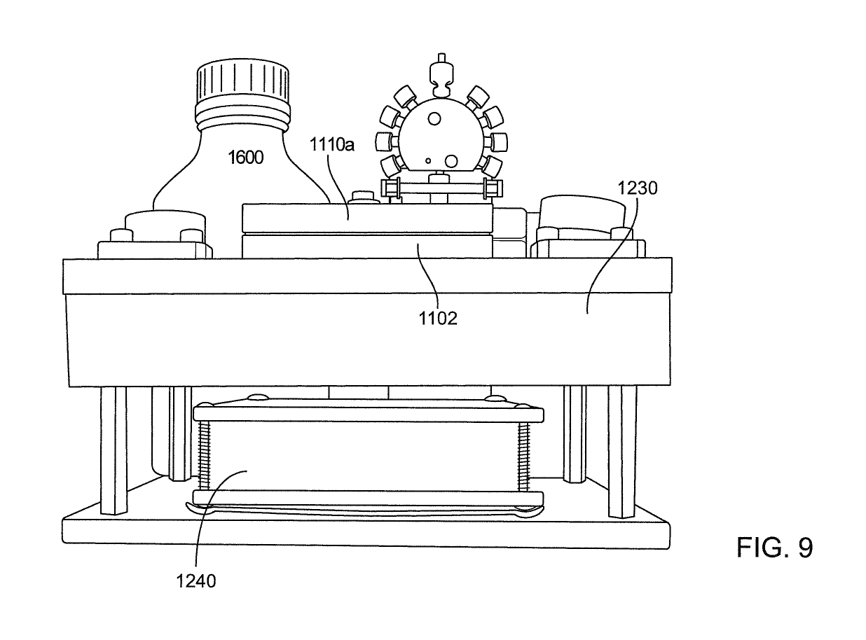

With additional reference to FIG. 9, each thermal regulation system 1200 can include one or more heating elements (not shown). Each heating element can be a resistive heater, a Peltier thermoelectric device, or the like, and can be thermally coupled to the metallic substrate 1150 of the mounting interface 1100 so as to control the temperature of a microfluidic device 100 securely mounted on a mounting interface 1100. The heating element can be enclosed in (or part of) a structure 1230 underlying the substrate 1150 of the mounting interface 1100. Such a structure 1230 can be metallic and/or configured to dissipate heat. For example, the structure 1230 can include metallic cooling vanes (best seen in FIGS. 6-8, on the adjacent culturing stations). Alternatively, or in addition, the thermal regulation system 1200 can include a heat dissipation device 1240, such as a fan (shown in FIG. 9) or a liquid-cooled cooling block (not shown), to help regulate the temperature of the heating element, and thereby regulate the temperature of the substrate 1150 of the mounting interface 110 and any microfluidic device 100 mounted thereon.

The thermal regulation system 1200 can further include one or more temperature sensors 1210 and, optionally, a temperature monitor 1250 (not shown) configured to display the temperature of the mounting interface 1100 or a microfluidic device 100 mounted thereon. The temperature sensors 1210 can be, for example, thermistors. The one or more temperature sensors 1210 can monitor the temperature of a microfluidic device 100 indirectly, by monitoring the temperature of a mounting interface 1100 on which the microfluidic device 100 is securely mounted. Thus, for example, the temperature sensor 1210 can be embedded in or otherwise thermally coupled to the metallic substrate 1150 of the mounting interface 1100. Alternatively, the temperature sensor 1210 can directly monitor the temperature of a microfluidic device 100, for example, by thermally coupling with a surface of the microfluidic device 100. As shown in FIGS. 6 and 7, the temperature sensor 1210 can directly contact the bottom surface of a microfluidic device 100 through an opening (or hole) in the substrate 1150 of the mounting interface 1100. As yet another alternative, which may be combined with any of the foregoing examples, the culturing station 1001/1002 can be operated with a microfluidic device 100 that includes a built-in temperature sensor (e.g., a thermistor), and the thermal regulation system 1200 can obtain temperature data from the microfluidic device 100. The thermal regulation system 1200 can thus measure the temperature of a microfluidic device 100 mounted on the mounting interface 1100. Regardless of how the temperature of the mounting interface 1100 and/or microfluidic device 100 is measured, the temperature data can be used by the thermal regulation system 1200 to regulate the heat produced by the one or more heating elements and, for systems that include a heat dissipation device 1240, the rate of dissipation of such heat.

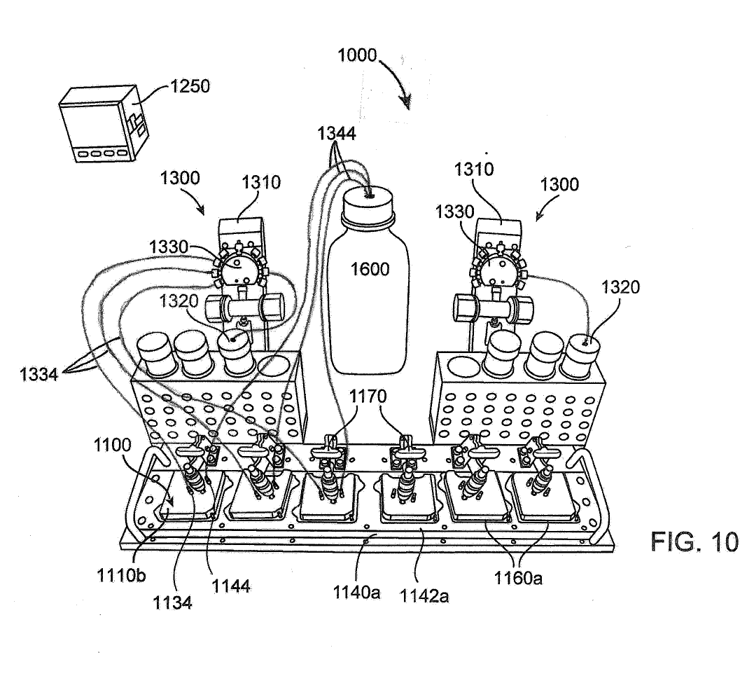

FIG. 10 depicts another embodiment of a culturing station, designated with reference number 1000, for culturing biological cells in microfluidic devices 100 (e.g., device 100 of FIGS. 1A-1C). In this embodiment, there are less pumps 1310 than mounting interfaces 1100, thus requiring that the pumps 1310 be configured to provide culturing media to multiple mounting interfaces 1100 (and the microfluidic devices 100 mounted thereon). As shown in FIG. 10, the culturing station 1000 can include one or more supports 1140 (labeled as 1140a in FIG. 10) each having a plurality (e.g., 2, 3, 4, 5, 6, 7, 8, 9, 10, or more) of thermally regulated microfluidic device mounting interfaces 1100, each mounting interface 1100 configured for having a microfluidic device 100 detachably mounted thereon. The support 1140a can be, for example, a tray.

Culturing stations such as culturing station 1000 shown in FIG. 10 can further include a thermal regulation system 1200 (not shown) configured for precisely controlling a temperature of each mounting interface 1100 and any microfluidic devices 100 detachably mounted thereon. The thermal regulation system 1200 can comprise a single heating element, which may be shared by two or more mounting interfaces 1100. Alternatively, the thermal regulation system 1200 may include two or more heating elements, each thermally coupled to a subset of mounting interfaces 1100 (e.g., the thermal regulation system 1200 can include a respective heating element for each mounting interface 1100, thereby allowing independent control of the temperature of each mounting interface 1100). As discussed above, each heating element may be a resistive heater, a Peltier thermoelectric device, or the like, and can be thermally coupled to at least one mounting interface 1100 of the support 1140a. For example, each heating element can be thermally coupled to at least one mounting interface 1100 (e.g., two or more, or all, mounting interfaces 1100) of the culturing station 1000. The heating element(s) can be thermally coupled to mounting interfaces via contact with a respective substrate 1150 of the mounting interfaces 1100. The thermal regulation system 1200 can also comprise at least one temperature sensor 1210 coupled to and/or embedded within support 1140a. As discussed above in connection with the culturing stations 1001/1002 of FIG. 5, the thermal regulation system 1200 can alternatively (or in addition) receive temperature data from a sensor coupled to and/or embedded within a microfluidic device 100. Regardless of the source of the temperature data, the thermal regulation system 1200 can use such data to regulate (e.g., increase or decrease) the amount of heat being produced by the heating element(s) and/or regulate a cooling device (e.g., a fan or a liquid-cooled cooling block).

Culturing stations such as culturing station 1000 shown in FIG. 10 can also include a media perfusion system 1300 configured to controllably and selectively dispense a flowable culturing media 1320 into microfluidic devices 100 securely mounted on one of the mounting interfaces 1100 of the support 1140a. The media perfusion system 1300 can include one or more (e.g., a pair of) pumps 1310, each pump 1310 having an input fluidically connected to a source of culturing media 1320. A respective multi-position valve 1330 selectively and fluidically connects an output of each pump 1310 with a plurality of perfusion lines 1334 associated with the mounting interfaces 1100. For example, as shown on the left-hand side of FIG. 10, each pump 1310 can be fluidically connected to perfusion lines 1334 associated with three respective mounting interfaces 1100. Perfusion lines 1334 (and waste lines 1344) were left out of the right-hand side of FIG. 10 for the sake of greater clarity, but it should be understood that a set of perfusion lines 1334 (and waste lines 1344) would typically be expected for both the right-hand and left-hand portions of the culturing station 1000 shown in FIG. 10. In addition, although three perfusion lines 1334 are shown in FIG. 10, there could be a different number (e.g., 2, 4, 5, 6, etc.). Thus, the media perfusion system 1300 could include a single pump 1310 that provides culturing media to all of the mounting interfaces 1100 (and the microfluidic chips 100 mounted on such mounting interfaces 1100) of the culturing station 1000 (or all of the mounting interfaces 1100 associated with a respective support 1140). Each perfusion line 1334 is configured to be fluidically connected to a fluid ingress port 124 of a microfluidic device 100 mounted on the respective mounting interface 1100 (the ingress port 124 on the device 100 shown in FIG. 10 is obscured by the below-described device cover). A control system (not shown) is configured to selectively operate the respective pumps 1310 and valves 1330 to thereby selectively cause culturing media from the culturing media source 1320 to flow through the respective perfusion lines 1334 at a controlled flow rate for a controlled period of time. More particularly, the control system is preferably programmed or may be programmed through operator input to provide an intermittent flow of the culturing media through the respective perfusion lines 1334 according to an on-off duty cycle and a flow rate. The on-off duty cycle and/or flow rate may be based at least in part on input received through a user interface (not shown). The control system is or may be programmed or otherwise configured to provide a flow of culturing medium through no more than a single perfusion line 1334 at any one time. For example, the control system can provide a flow of culturing medium to each of the perfusion lines 1334 in series, as discussed further below. The control system may alternatively be programmed or otherwise configured to provide a flow of culturing media through two or more perfusion lines 1334 at the same time.

In various embodiments, the flow of culturing media to the flow region of the microfluidic circuit 134 of a microfluidic device 100 mounted on a mounting interface 1100 of an exemplary culturing station (e.g., culturing station 1000/1001/1002) preferably occurs periodically for about 10 seconds to about 120 seconds. Other “flow ON” time periods may also be used, including the following ranges: from about 10 seconds to about 20 seconds; from about 10 seconds to about 30 seconds; from about 10 seconds to about 40 seconds; from about 20 seconds to about 30 seconds; from about 20 seconds to about 40 seconds; from about 20 seconds to about 50 seconds; from about 30 seconds to about 40 seconds; from about 30 seconds to about 50 seconds; from about 30 seconds to about 60 seconds; from about 45 seconds to about 60 seconds; from about 45 seconds to about 75 seconds; from about 45 seconds to about 90 seconds, from about 60 seconds to about 75 seconds; from about 60 seconds to about 90 seconds; from about 60 seconds to about 105 seconds; from about 75 seconds to about 90 seconds; from about 75 seconds to about 105 seconds; from about 75 seconds to about 120 seconds; from about 90 seconds to about 120 seconds; from about 90 seconds to about 150 seconds; from about 90 seconds to about 180 seconds; from about 2 minutes to about 3 minutes; from about 2 minutes to about 5 minutes; from about 2 minutes to about 8 minutes; from about 5 minutes to about 8 minutes; from about 5 minutes to about 10 minutes; from about 5 minutes to about 15 minutes; from about 10 minutes to about 15 minutes; from about 10 minutes to about 20 minutes; from about 10 minutes to about 30 minutes; from about 20 minutes to about 30 minutes; from about 20 minutes to about 40 minutes; from about 20 minutes to about 50 minutes; from about 30 minutes to about 40 minutes; from about 30 minutes to about 50 minutes; from about 30 minutes to about 60 minutes; from about 45 minutes to about 60 minutes; from about 45 minutes to about 75 minutes; from about 45 minutes to about 90 minutes; from about 60 minutes to about 75 minutes; from about 60 minutes to about 90 minutes; from about 60 minutes to about 105 minutes; from about 75 minutes to about 90 minutes; from about 75 minutes to about 105 minutes; from about 75 minutes to about 120 minutes; from about 90 minutes to about 120 minutes; from about 90 minutes to about 150 minutes; from about 90 minutes to about 180 minutes; from about 120 minutes to about 180 minutes; and from about 120 minutes to about 240 minutes.

In other embodiments, the flow of culturing media to the flow region of the microfluidic circuit 134 of a microfluidic device 100 mounted on a mounting interface 1100 of an exemplary culturing e station (e.g., culturing station 1000/1001/1002) is stopped periodically for about 5 seconds to about 60 minutes. Other possible “flow OFF” ranges include: from about 5 minutes to about 10 minutes; from about 5 minutes to about 20 minutes; from about 5 minutes to about 30 minutes; from about 10 minutes to about 20 minutes; from about 10 minutes to about 30 minutes; from about 10 minutes to about 40 minutes; from about 20 minutes to about 30 minutes; from about 20 minutes to about 40 minutes; from about 20 minutes to about 50 minutes; from about 30 minutes to about 40 minutes; from about 30 minutes to about 50 minutes; from about 30 minutes to about 60 minutes; from about 45 minutes to about 60 minutes; from about 45 minutes to about 75 minutes; from about 45 minutes to about 90 minutes; from about 60 minutes to about 75 minutes; from about 60 minutes to about 90 minutes; from about 60 minutes to about 105 minutes; from about 75 minutes to about 90 minutes; from about 75 minutes to about 105 minutes; from about 75 minutes to about 120 minutes; from about 90 minutes to about 120 minutes; from about 90 minutes to about 150 minutes; from about 90 minutes to about 180 minutes; from about 120 minutes to about 180 minutes; from about 120 minutes to about 240 minutes; and from about 120 minutes to about 360 minutes.

In some embodiments, the control system of the media perfusion system 1300 can be programmed to perform a multi-step process comprising the steps of: providing culturing medium (or “perfusing”) a first microfluidic device 100 securely mounted on a mounting interface 1100 for a first period of time while providing no culturing medium for a second and a third microfluidic device 100, each also securely mounted on a mounting interface 1100; perfusing the second microfluidic device 100 for a second period of time (which can be equal to the first period of time) while providing no culturing medium to the first and third microfluidic devices 100; perfusing the third microfluidic device 100 for a third period of time (which can be equal to the first and/or second period of time) while providing no culturing medium for the first and second microfluidic devices 100; and repeating the foregoing set of steps n times, wherein n equals 0 or a positive integer. Each time the first three steps are performed can be considered a “cycle” or “duty cycle” during which each of the first, second, and third microfluidic devices 100 experience a period of “flow ON” and a period of “flow OFF.” If each of the first, second, and third time periods are all equal to 60 seconds, then each microfluidic device 100 will experience a duty cycle of 33% for a duration of 3 minutes. As the number of microfluidic being perfused by a single pump 1310 of the media perfusion system 1300 increases, the duty cycle will decrease and the duration will increase. In some embodiments, the on-off duty cycle may have a total duration of about 3 minutes to about 60 minutes (e.g., about 3 minutes to about 6 minutes, about 4 minutes to about 8 minutes, about 5 minutes to about 10 minutes, about 6 minutes to about 12 minutes, about 7 minutes to about 14 minutes, about 8 minutes to about 16 minutes, about 9 minutes to about 18 minutes, about 10 minutes to about 20 minutes, about 15 minutes to about 20, 25, or 30 minutes, or about 30 minutes to about 40, 50, or 60 minutes). In alternate embodiments, the on-off duty cycle can vary anywhere from about 5 minutes to about 4 hours. In some embodiments, the foregoing process can be performed for n=0, 1, 2, 3, 4, 5, 6, 7, 8, 9, 10, 15, 20, 25, 30, 35, 40, 45, 50, or more repetitions. Thus, the total duration of the process can take hours or days, depending upon the total duration of each duty cycle. Furthermore, the process, once finished, can be immediately started with a new duty cycle. For example, a first duty cycle could include a relatively slow rate of perfusion (e.g., about 0.001 microliters/sec to about 0.01 microliters/sec) and a second duty cycle could include a relative fast rate of perfusion (e.g., greater than about 0.1 microliters/sec). Such alternate duty cycles could be performed repeatedly (e.g., cycle 1 followed by cycle 2, then repeat).

Culturing medium can be flowed through the flow region of a microfluidic device 100 according to a predetermined and/or operator selected flow rate, wherein the flow rate is about 0.01 microliters/sec to about 5.0 microliters/sec. Other possible ranges include about 0.001 microliters/sec to about 1.0 microliters/sec, about 0.005 microliters/sec to about1.0 microliters/sec, about 0.01 microliters/sec to about 1.0 microliters/sec, about 0.02 microliters/sec to about 2.0 microliters/sec, about 0.05 microliters/sec to about1.0 microliters/sec, about 0.08 microliters/sec to about 1.0 microliters/sec, about 0.1 microliters/sec to about 1.0 microliters/sec, about 0.1 microliters/sec to about 2.0 microliters/sec, about 0.2 microliters/sec to about 2.0 microliters/sec, about 0.5 microliters/sec to about 2.0 microliters/sec, about 0.8 microliters/sec to about 2.0 microliters/sec, about 1.0 microliters/sec to about 2.0 microliters/sec, about 1.0 microliters/sec to about 5.0, about 1.5 microliters/sec to about 5.0 microliters/sec, about 2.0 microliters/sec to about 5.0 microliters/sec, about 2.5 microliters/sec to about 5.0 microliters/sec, about 2.5 microliters/sec to about 10.0 microliters/sec, about 3.0 microliters/sec to about 10.0 microliters/sec, about 4.0 microliters/sec to about 10.0 microliters/sec, about 5.0 microliters/sec to about 10.0 microliters/sec, about 7.5 microliters/sec to about 10.0 microliters/sec, about 7.5 microliters/sec to about 12.5 microliters/sec, about 7.5 microliters/sec to about 15.0 microliters/sec, about 10.0 microliters/sec to about 15.0 microliters/sec, about 10.0 microliters/sec to about 20.0 microliters/sec, about 10.0 microliters/sec to about 25.0 microliters/sec, about 15.0 microliters/sec to about 20.0 microliters/sec, about 15.0 microliters/sec to about 25.0 microliters/sec, about 15.0 microliters/sec to about 30.0 microliters/sec, about 20.0 microliters/sec to about 30.0 microliters/sec, about 20.0 microliters/sec to about 40.0 microliters/sec, about 20.0 microliters/sec to about 50.0 microliters/sec microliters/sec.

As discussed above, the flow region of the microfluidic circuit in a microfluidic device 100 can comprises two or more flow channels. Thus, the rate of flow of medium through each individual channel is expected to be about 1/m the rate of flow of medium through the entire microfluidic device, wherein m=the number of channels in the microfluidic device 100. In certain embodiments, culturing medium can be flowed through each of the two or more flow channels an average rate of about 0.005 microliters/sec to about 2.5 microliters/sec. Additional ranges are possible and can be, for example, readily calculated as 1/m times the endpoints of the ranges disclosed herein.

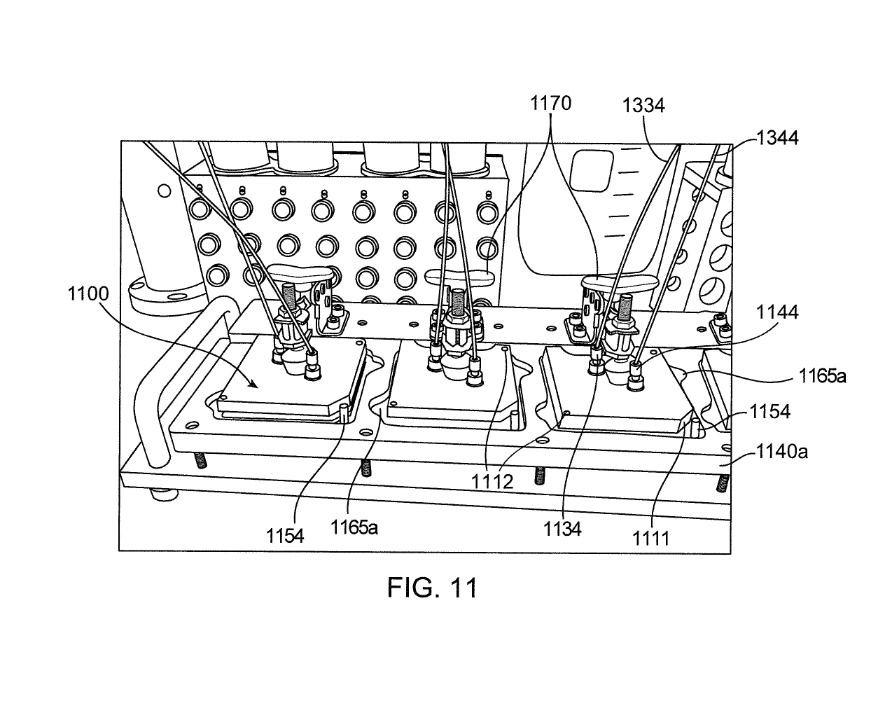

With reference to the culturing station embodiments shown in FIGS. 10 and 11, each microfluidic device mounting interface 1100 can include a microfluidic device cover 1110 (identified as 1110b) configured to at least partially enclose a microfluidic device 100 mounted on the respective mounting interface 1100 of the support 1140a. The microfluidic device covers 1110b can be secured (e.g., each by a respective clamp 1170, as shown) to the respective mounting interfaces 1100, each enclosing a respective microfluidic device 100. Distal end connectors 1134 for respective perfusion lines 1334 associated with the mounting interfaces 1100 can be coupled to the microfluidic device covers 1110b, and the microfluidic device covers 1110b can be configured so that the respective perfusion lines 1334 are fluidically connected to the fluid ingress port 124 of a mounted microfluidic device 100 when the microfluidic device 100 is enclosed (e.g., properly positioned and securely held) by the respective microfluidic device cover 1110b. By way of example, each microfluidic device cover 1110b may include one or more features configured to form a pressure fit, a frictional fit, or another type of fluid tight connection between the distal end of the respective perfusion line 1334 and the respective fluid ingress port 124 of the microfluidic device 100, in order to fluidically connect the perfusion line 1334 to the microfluidic circuit 134 of the device 100. The microfluidic device covers 1110b of FIGS. 10-12 and 14 have no windows, and thus are alternative covers that may be used in place of the device covers 1110a that include windows 1104, as shown in FIG. 8. However, the microfluidic device covers 1110b of FIGS. 10-12 and 14 could be readily designed to include a window (e.g., if imaging of the microfluidic device 100 is desired during culture).

A respective waste line 1344 can be associated with each mounting interface 1100. For example, each waste line 1344 can be connected to a respective microfluidic device cover 1110b via a proximal end connector 1144. Thus, the waste lines 1344 can be configured, in conjunction with a configuration of the microfluidic device covers 1110b, so that the proximal ends of the waste lines 1440 are fluidically connected to a fluid egress port 124 (obscured by the cover 1110b in FIG. 11) on the microfluidic device 100 when the microfluidic device 100 is enclosed (e.g., properly positioned and securely held, such as by clamps 1170) by the microfluidic device cover 1110b. By way of example, each microfluidic device cover 1110b may include one or more features configured to form a pressure fit, a frictional fit, or another type of fluid tight connection between the distal end of the respective waste line 1344 and the respective fluid egress port 124 of the microfluidic device 100, in order to fluidically connect the waste line 1344 to the microfluidic circuit 134 of the device 100. The distal end of each waste line can be connected and/or fluidically coupled to a waste container 1600.

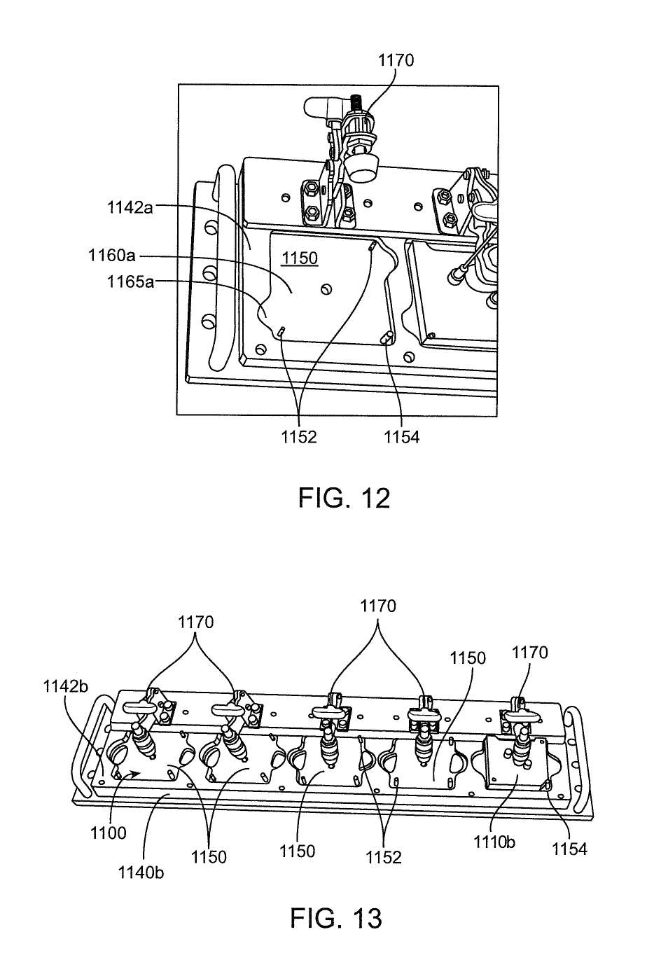

With additional reference to FIG. 12, each mounting interface 1100 can comprise a metallic substrate 1150, which may have a generally planar top surface configured to thermally couple with a generally planar metallic bottom surface (not shown) of a microfluidic device 100 mounted thereon. The support 1140a can include a top surface 1142a having a plurality of windows 1160a (e.g., six windows 1160a, as shown in FIG. 10, though the number may be small or larger) exposing the respective metallic substrates 1150. In addition, the top surface 1142a of the tray 1140a can be shaped and sized to form openings 1165a (FIG. 11) configured to facilitate placement and/or retrieval of microfluidic devices 100 from the mounting interfaces 1100 by a user (e.g., by placing fingers in openings 1165a). As shown, the openings 1165a in the top surface 1142a of the support 1140a can be diagonally disposed relative to each other in each window 1160a.

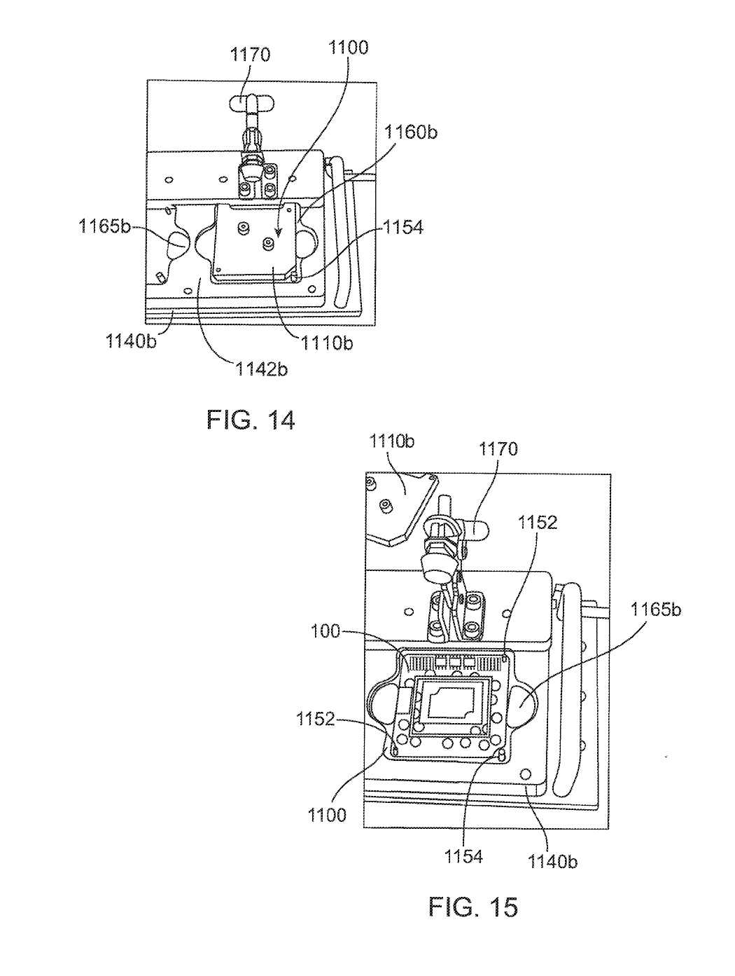

With further reference to FIGS. 11-15, each mounting interface 1100 can comprise an alignment pin 1154 configured to assist the user with a proper orientation and placement of the microfluidic device 100 and/or the microfluidic device cover 1110b within the respective window 1160a of a mounting interface 1100. The alignment pin 1154 can be disposed on the substrate 1150, usually at a corner of the window 1160a/1160b. Each corresponding device cover 1110b can further comprise an orientation element 1111, such as a tapered end corner (better seen in FIGS. 11 and 14), a loop, hook, or the like, configured to meet, engage and/or face the respective alignment pin 1154, and further assist the user with the proper orientation and placement of the device cover 1110b within the respective window 1160a/1160b in the mounting interface 1100.

Each mounting interface 1100 can further comprise additional alignment features. As shown in FIGS. 12 and 15, in which the microfluidic device cover 1110b has been removed to clearly expose the mounting interface 1100, one or more engagement pins 1152 (e.g., two are shown, but the number can be more than two or less than two) can be used to further assist with the proper placement of the microfluidic device 100 and/or the device cover 1110b within the respective window 1160a/1160b of the mounting interface 1100. As can be see, the engagement pins 1152 can be disposed on the metallic substrate 1150, at opposite corners of the respective window 1160a/1160b (i.e., diagonally disposed relative to each other). The engagement pins 1152 are configured to meet and engage with a respective pair of engagement openings 1112 in the microfluidic device cover 1110b (FIG. 14), and with a respective pair of engagement openings 113 of the microfluidic device 100 (FIG. 15). The pair of engagement openings 1112 are disposed at opposite corners of the respective microfluidic device cover 1110b (or diagonally disposed relative to each other), as better seen in FIGS. 11 and 13. The pair of engagement openings 113 of microfluidic device 100 are disposed at opposite corners of the device 100 (or diagonally disposed relative to each other), as better seen in FIG. 15.

Those skilled in the art will appreciate that various arrangements and configurations of the alignment pin 1154 and/or the engagement pins 1152 of the mounting interface 1100, the orientation element 1111 and engagement openings 1112 of the microfluidic device cover 1110b, and the engagement openings 113 of the microfluidic device 100 can be used to achieve the goal of facilitating proper alignment of the microfluidic device 100 and/or the microfluidic device cover 1110b. By way of example, the alignment pin 1154 and the engagement pins 1152 can have a variety of shapes including but not limited to: a circular, oval, rectangular, cylindrical (as shown), or multi-sided shape, or irregular shapes and/or angles that are adapted to meet and engage with the corresponding orientation element 1111 and engagement openings 1112 and 113, respectively.

FIG. 13 illustrates an alternate support 1140 (labeled as 1140b to distinguish it from the support 1140a of FIG. 10) that can be used in an exemplary culturing station (e.g., culturing station 1000). The support includes five thermally regulated mounting interfaces 1100 and can replace the support 1140a of the culturing station 1000 shown in FIG. 10. It will be appreciated that the support 1140b may be used with a media perfusion system 1300 having a single pump 1310 or multiple pumps 1310 (e.g., two, as shown in FIG. 10). Moreover, the culturing station 1000 can comprise two or more supports 1140a/1140b, each of which may be associated with a respective pump 1310. The tray 1140b includes a top surface 1142b having five windows 1160b exposing the respective metallic substrates 1150. For illustration purposes, FIG. 13 shows four of the five windows 1160b exposing their respective substrate 1150; the substrate 1150 and respective microfluidic device 100 of the fifth window 1160b (on the right) are covered by a microfluidic device cover 1110b. The top surface 1142b of the tray 1140b is shaped and sized to form respective openings 1165b configured to facilitate placement and/or retrieval of microfluidic devices 100 by an user (e.g., by placing fingers in openings 1165b). The openings 1165b on the top surface 1142b of the tray 1140b can be disposed in various relative orientations, including being parallel relative to each other in each window 1160b, as shown in FIGS. 13-15.

It will be appreciated that, when in use, the thermally regulated mounting interfaces 1110b of FIG. 13 would include respective microfluidic device covers 1110b configured to secure respective mounting interfaces 1100, each enclosing a respective mounted microfluidic device 100. The securing mechanism for the microfluidic device covers 1110b can be a clamp 1170, as shown in FIGS. 10-15. However, any suitable securing mechanism could be used in place of the clamp 1170, including, for example, screws (as discussed in connection with the microfluidic device covers 1110a of the culturing station 1001/1002) optionally in combination with compression springs.

FIG. 14 illustrates one of the thermally regulated mounting interfaces 1100 of the tray 1140b shown in FIG. 13, depicting a microfluidic device cover 1110b. Each microfluidic device cover 1110b is configured to at least partially enclosed a microfluidic device 100 mounted on the respective mounting interface 1100 of the tray 1140b. The device cover 1110b is disposed within a respective window 1160b formed by the top surface 1142b of the tray 1140b. In this embodiment, the device cover 1110b is unsecured (i.e., respective clamp 1170 is unengaged) to allow placement and/or retrieval of the device cover 1110b and microfluidic device 100 by a user (e.g., by placing fingers in openings 1165b).

FIG. 15 illustrates the mounting interface 1100 of FIG. 14, having the microfluidic device cover 1110b removed from the mounting interface 1100 to show the microfluidic device 100 mounted thereon. The removed microfluidic device cover 1110b exposes the microfluidic device 100 mounted on the respective mounting interface 1100, and further exposes engagement pins 1152. The top surface 1142a of the tray 1140a is shaped and sized to form respective openings 1165b configured to allow placement and/or retrieval of the microfluidic device 100 from the respective window 1160a (e.g., by placing fingers in openings 1165b).

Each culturing station 1000 of the invention can additionally be configured to record in a memory respective perfusion and/or temperature histories of microfluidic devices 100 mounted to the one or more mounting interfaces 1100. For example, the culturing station may include a processor and memory, either or both of which may be integrated into a printed circuit board. Alternatively, the memory may be incorporated into or otherwise coupled with the respective microfluidic device 100. The culturing stations 1000 may additionally (optionally) including an imaging and/or detecting apparatus (not shown) coupled to or otherwise operatively associated with the culturing stations 1000 and configured for viewing and/or imaging micro-objects within a microfluidic device 100 and/or detecting biological activity in the microfluidic device 100 mounted to one of the mounting interfaces 1100. The resulting data may be processed and/or stored in memory located within the culturing station 1000 and/or the microfluidic device 100, as discussed above.

An exemplary culturing station, such as culturing station 1000, can also be configured to allow mounting interfaces 1100 to be tilted upon an axis, such that a microfluidic device 100 mounted on the mounting interface 1100 can be optimally positioned for culturing. In some embodiments, a microfluidic device 100 can be tilted, for example, relative to a plane that is normal to the force of gravity acting upon the culturing station 1000, by about 1° to about 10° (e.g., about 1° to about 5° , or about 1° to about 2°). Alternatively, the mounting interfaces 1100 can be configured to be tilted to at least about 45°, 60°, 75°, 90°, or ever further (e.g., at least about 105°, 120°, or) 135°. In some embodiments, a plurality of mounting interfaces 1100 can be tilted simultaneously upon a common access. For example, the support 1140a/1140b of any of FIGS. 10-15 could be configured to rotate around an axis (e.g., a long axis) such that each mounting interface on the support 1140a/1140b is tilted at the same time. Whether the mounting interfaces 1100 tilt individually or as a group, it can be desirable to lock the tilted mounting interfaces into a specific position (e.g., with the microfluidic devices 100 mounted on the mounting interfaces 1100 positioned vertically). Thus, the mounting interfaces 1100 or the support 1140a/1140b can include a locking element to hold the mounting interfaces 1100 in a tilted position. To facilitate positioning the mounting interfaces 1100 at a specific degree of tilt, a level can be mounted on the mounting interface 1100 or a surface 1142a/1142b of the support 1140a/1140b comprising the mounting interface 1100. For example, the level can be mounted in such a manner that it is “level” (i.e., parallel to a plane normal to the force of gravity acting upon the culturing station 1000) only when the mounting interface 1100 or support 1140a/1140b is tilted to a predetermined degree.

While embodiments have been shown and described, various modifications may be made without departing from the scope of the inventive concepts disclosed herein. The invention(s), therefore, should not be limited, except as defined in the following claims.

1. A culturing station for culturing biological cells in a microfluidic device, comprising:
a plurality of thermally conductive mounting interfaces, each mounting interface configured for having a microfluidic device detachably mounted thereon;
a thermal regulation system configured for controlling a temperature of microfluidic devices detachably mounted on the one or more mounting interfaces; and
a media perfusion system configured to controllably and selectively dispense a flowable culturing media into a microfluidic device detachably mounted on any one of the plurality of mounting interfaces.

2. (canceled)

3. The culturing station of claim 1, wherein the plurality of mounting interfaces comprises at least three mounting interfaces.

4. The culturing station of claim 1, wherein the media perfusion system comprises
a pump having an input fluidically connected to a source of culturing media and an output;
a perfusion network that fluidically connects the pump output with a plurality of perfusion lines, each perfusion line configured to be fluidically connected to a fluid ingress port of a microfluidic device mounted on a respective mounting interface; and
a control system configured to selectively operate the pump and the perfusion network to thereby selectively cause culturing media from the culturing media source to flow through one of the plurality of perfusion lines at a controlled flow rate for a controlled period of time,
wherein the control system is programmed or otherwise configured to provide an intermittent flow of culturing media through a respective perfusion line according to an on-off duty cycle and a flow rate.

5. (canceled)

6. The culturing station of claim 4, wherein the on-off duty cycle and/or flow rate are based at least in part on input received through a user interface.

7. The culturing station of claim 4, wherein the control system is programmed or otherwise configured to provide a flow of culturing media through no more than a single perfusion line at any one time.

8. (canceled)

9. The culturing station of claim 1, further comprising a plurality of microfluidic device covers, each microfluidic device cover of the plurality configured to at least partially enclose a microfluidic device mounted on a respective mounting interface.

10. The culturing station of claim 4 further comprising
a plurality of microfluidic device covers, each microfluidic device cover of the plurality configured to at least partially enclose a microfluidic device mounted on a respective mounting interface; and
a plurality of waste lines, each waste line associated with a respective mounting interface,
wherein each of the plurality of perfusion lines has a distal end coupled to the microfluidic device cover associated with the respective mounting interface, and is configured, in conjunction with a configuration of the device cover, so that the distal end of the perfusion line may be fluidically connected to a fluid ingress port on a microfluidic device mounted on the mounting interface and enclosed by the microfluidic device cover, and
wherein each of the plurality of waste lines has a proximal end coupled to the microfluidic device cover associated with the respective mounting interface, and is configured, in conjunction with a configuration of the microfluidic device cover, so that the proximal end of the waste line may be fluidically connected to a fluid egress port on a microfluidic device mounted on the mounting interface and enclosed by the microfluidic device cover.

11. The culturing station of claim 10, wherein each microfluidic device cover includes one or more features configured to form a pressure fit, a frictional fit, or another type of fluid tight connection between the distal end of the respective perfusion line and the fluid ingress port of the microfluidic device in order to fluidically connect the perfusion line to the microfluidic device.

12-13. (canceled)

14. The culturing station of claim 10, wherein each microfluidic device cover includes one or more features configured to form a pressure fit, a frictional fit, or another type of fluid tight connection between the proximal end of the respective waste line and the fluid egress port of the microfluidic device, in order to fluidically connect the waste line to the microfluidic device.

15-16. (canceled)

17. The culturing station of claim 1, the thermal regulation system comprising one or more resistive heaters thermally coupled to each of the plurality of mounting interfaces.

18. The culturing station of claim 17, wherein each of the one or more resistive heaters is thermally coupled to a respective one of the plurality of mounting interfaces.

19-20. (canceled)

21. The culturing station of claim 1, wherein each of the plurality of mounting interfaces comprises a generally planar metallic substrate having a top surface configured to thermally couple with a generally planar metallic bottom surface of a microfluidic device mounted thereon.

22. The culturing station of claim 21, the generally planar metallic substrate having a bottom surface configured to thermally couple with a resistive heater of the thermal regulation system.

23. (canceled)

24. The culturing station of claim 21, the thermal regulation system comprising a plurality of temperature sensors, each temperature sensor coupled to and/or embedded within a respective mounting interface substrate and configured to monitor a temperature of the respective substrate of the respective mounting interface.

25. (canceled)

26. The culturing station of claim 21, the thermal regulation system configured to obtain temperature data from one or more temperature sensors coupled to and/or embedded within each microfluidic device mounted on a mounting interface.

27. (canceled)

28. The culturing station of claim 9, further comprising a plurality of adjustable clamps and/or a plurality of compression springs,
wherein each clamp is positioned adjacent a respective one of the plurality of mounting interfaces and is configured to apply a force against the microfluidic device cover associated with the mounting interface, such that the microfluidic device cover secures a microfluidic device at least partially enclosed by the microfluidic device cover to the respective mounting surface, and
wherein each compression spring is associated with a respective one of the plurality of mounting interfaces and is configured to apply a force against the microfluidic device cover associated with the mounting interface, such that the microfluidic device cover secures a microfluidic device at least partially enclosed by the microfluidic device cover to the respective mounting surface.

29. The culturing station of claim 10, further comprising a plurality of adjustable clamps and/or a plurality of compression springs,
wherein each clamp is positioned adjacent a respective one of the plurality of mounting interfaces and is configured to apply a force against the microfluidic device cover associated with the mounting interface, such that the microfluidic device cover secures a microfluidic device at least partially enclosed by the microfluidic device cover to the respective mounting surface, and
wherein each compression spring is associated with a respective one of the plurality of mounting interfaces and is configured to apply a force against the microfluidic device cover associated with the mounting interface, such that the microfluidic device cover secures a microfluidic device at least partially enclosed by the microfluidic device cover to the respective mounting surface.

30. The culturing station of claim 1, wherein the culturing station is configured to record in a memory respective perfusion and/or temperature histories of a microfluidic device mounted on one of the plurality of mounting interfaces.

31. The culturing station of claim 30, wherein the memory is incorporated into or otherwise coupled with the respective microfluidic device.

32. The culturing station of claim 1, further comprising a level configured to indicate when the one or more mounting interfaces are tilted relative to a plane that is normal to a gravitational force acting upon the culturing station.

33. The culturing station of claim 32, the level indicating when the one or more mounting interfaces is/are tilted at a pre-determined degree relative to the normal plane, wherein the pre-determined degree of tilt is within a range of about 1° to about 5°.

34. (canceled)

35. The culturing station of claim 1, further comprising an imaging and/or detecting apparatus coupled to or otherwise operatively associated with the culturing station and configured for viewing and/or imaging and/or detecting biological activity in a microfluidic device mounted on one of the one or more mounting interfaces.

36. A method for culturing biological cells in a microfluidic device, comprising:
mounting a microfluidic device on a mounting interface of a culturing station, the microfluidic device defining a microfluidic circuit including a flow region and a plurality of growth chambers, the microfluidic device comprising a fluid ingress port in fluid communication with a first end region of the microfluidic circuit, and a fluid egress port in fluid communication with a second end region of the microfluidic circuit;
fluidically connecting a perfusion line associated with the mounting interface to the fluid ingress port to thereby fluidically connect the perfusion line with the first end region of the microfluidic circuit;
fluidically connecting a waste line associated with the mounting interface to the fluid egress port to thereby fluidically connect the waste line with the second end region of the microfluidic circuit; and
flowing a culturing media through the perfusion line, fluid ingress port, flow region of the microfluidic circuit, and fluid egress port, respectively, at a flow rate adequate to perfuse one or more biological cells sequestered in the plurality of growth chambers,
wherein flowing the culturing media comprises providing an intermittent flow of culturing media through the flow region of the microfluidic circuit.

37. (canceled)

38. The method of claim 36, wherein the culturing media is flowed through the flow region of the microfluidic circuit according to a predetermined and/or operator selected on-off duty cycle.

39. The method of claim 38, wherein the flow of culturing media in the flow region of the microfluidic circuit occurs periodically for about 10 seconds to about 120 seconds,
wherein the flow of culturing media in the flow region of the microfluidic circuit is stopped periodically for about 30 seconds to about 30 minutes, and
wherein the on-off duty cycle has a total duration of about 5 minutes to about 30 minutes.

40-41. (canceled)

42. The method of claim 36, wherein the culturing media is flowed through the flow region of the microfluidic circuit according to a predetermined and/or operator selected flow rate of about 0.01 microliters/sec to about 5.0 microliters/sec.

43-46. (canceled)

47. The method of claim 36, further comprising controlling a temperature of the microfluidic device using at least one heating element that is thermally coupled to the mounting interface.

48-49. (canceled)

50. The method of claim 36, further comprising recording perfusion and/or temperature histories of the microfluidic device while it is mounted to the mounting interface.

51. (canceled)
